(19) United States
(12) Patent Application Publication
(10) Pub. No.: US 2012/0196279 A1
(43) Pub. Date: Aug. 2, 2012

(54) METHODS AND COMPOSITIONS FOR NUCLEIC ACID SAMPLE PREPARATION

(75) Inventors: Jason Underwood, Redwood City, CA (US); Swati Ranade, Foster City, CA (US); Yu-Chih Tsai, Mountain View, CA (US)

(73) Assignee: Pacific Biosciences of California, Inc., Menlo Park, CA (US)

(21) Appl. No.: 13/363,066

(22) Filed: Jan. 31, 2012

Related U.S. Application Data
(60) Provisional application No. 61/438,860, filed on Feb. 2, 2011.

(51) Int. Cl. C12Q 1/68; C12P 19/34

(52) U.S. Cl. 435/6.1; 435/91.51; 435/91.52

(57) ABSTRACT

Provided are methods and compositions for the production of double-stranded nucleic acids, which can optionally be used as templates in high-throughput sequencing systems. In certain embodiments, these templates do not require exogenous primers to facilitate initiation of polymerase-dependent nascent strand synthesis. In certain embodiments, these templates comprise a single-stranded or gapped region that serves as a polymerase priming site.

This application claims the benefit of U.S. Provisional Application No. 61/438,860, filed Feb. 2, 2011, the full disclosure of which is incorporated herein by reference in its entirety for all purposes.

### BACKGROUND OF THE INVENTION

Nucleic acid sequence data is valuable in myriad applications in biological research and molecular medicine, including determining the hereditary factors in disease, in developing new methods to detect disease and guide therapy (van de Vijver et al. (2002) “A gene-expression signature as a predictor of survival in breast cancer,” New England Journal of Medicine 347: 1999-2009), and in providing a rational basis for personalized medicine. Obtaining and verifying sequence data for use in such analyses has made it necessary for sequencing technologies to undergo advancements to expand throughput, lower reagent and labor costs and improve accuracy (See, e.g., Chan, et al. (2005) “Advances in Sequencing Technology” (Review) Mutation Research 573: 13-40, Levene et al. (2003) “Zero Mode Waveguides for Single Molecule Analysis at High Concentrations,” Science 299: 682-686).

Current methods for preparing nucleic acid templates are not optimal for use in high throughput DNA sequencing systems. Conventional cloning and cell culture methods are time consuming and expensive. Lengthy nucleic acid purification protocols currently in use do not reliably produce nucleic acid samples that are sufficiently free of sequencing reaction inhibitors such as salts, carbohydrates and/or proteins. Furthermore, these problems are magnified when such conventional techniques are scaled to the quantities that would be useful for high throughput sequencing technologies. Consequently, there is an increasing demand for efficient, low-cost methods for the preparation of high-quality nucleic acid templates. The present invention provides methods and compositions that would be useful for supplying high throughput DNA sequencing systems with such templates.

### SUMMARY OF CERTAIN ASPECTS OF THE INVENTION

The present invention provides methods and compositions that can be useful for supplying high throughput nucleic acid sequencing systems with templates. The methods circumvent the need for costly, labor-intensive cloning and cell culture methods and can be scaled to accommodate template production for a variety of sequencing applications, e.g., sequencing individuals' genomes and/or gene expression profiling (Spinella, et al. (1999) “Tandem arrayed ligation of expressed sequence tags (TALEST): a new method for generating global gene expression profiles.” Nucleic Acids Res 27: e22, Velculescu, et al. (1995) “Serial analysis of gene expression.” Science 270: 484-487). The methods and compositions provided by the invention can be used to produce either linear or circular single-stranded nucleic acid templates.

In certain aspects, the invention provides a first set of methods of producing a population of double-stranded DNA templates that can be subjected to template-directed sequencing reactions in the absence of any exogenous priming oligos. A genomic DNA, a cDNA, or a DNA concatamer is provided, e.g. from a eukaryote, a prokaryote, an archaea, a virus, or a phage. Generating the double-stranded fragments can optionally comprise cleaving the genomic DNA, cDNA, or concatamer, e.g., via enzymatic digestion, sonication, mechanical shearing, electrochemical cleavage, and/or nebulization. In certain preferred embodiments, the double-stranded. DNA templates are subjected to amplification using at least one chimeric primer having an RNA region. Optionally a second primer is also used to allow for exponential amplification, and the second primer may or may not comprise an RNA region. Following amplification, the resulting amplicons are subjected to treatment with an RNA-degrading enzyme, e.g., RNaseH, and this treatment results in gaps that serve as polymerase priming sites during the subsequence template-directed sequencing reaction. Optionally, primer-free binding and initiation sites can be introduced by nicking at a pre-determined location in the primer. In certain embodiments, both primers used in amplification have a region that can be modified to allow binding and initiation of nascent strand synthesis.

In certain aspects of the present invention, methods are provided for producing a double-stranded nucleic acid having a single-stranded region. In certain embodiments, such methods comprise providing a double-stranded DNA molecule; fragmenting the double-stranded. DNA molecule to produce double-stranded DNA fragments; attaching polynucleotides to the ends of the double-stranded DNA fragments, wherein at least one of the polynucleotides on each of the fragments comprises a region of ribonucleotides; and eliminating the region of ribonucleotides to produce a double-stranded nucleic acid having a single-stranded region. In some embodiments, the eliminating is performed using a ribonuclease, e.g., RNaseH. In certain embodiments, the attaching comprises performing a single-step primer extension from a primer comprising the region of ribonucleotides or ligating an adapter comprising the region of ribonucleotides. Optionally, i) the adapter can further comprise a region of deoxyribonucleotides that is terminally located after the attaching, or ii) the adapter can be a single-stranded adapter that is ligated to 5′ ends of both strands of the double-stranded DNA fragments, and the method further comprises performing a strand extension reaction to extend 3′ ends of both strands of the double-stranded DNA fragments, thereby converting the single-stranded adapter to a double-stranded adapter. In some embodiments, the fragmenting comprises one or more of: enzymatic digestion, sonication, mechanical shearing, electrochemical cleavage, or nebulization.

In further aspects, the present invention provides methods for performing template-directed nascent strand synthesis. In certain embodiments, such methods comprise producing a double-stranded nucleic acid molecule having a single-stranded region, exposing the nucleic acid molecule to a polymerase enzyme in the presence of nucleotides, and monitoring incorporation of the nucleotides into a nascent strand complementary to the nucleic acid molecule. In certain embodiments, the polymerase enzyme is a type A or type B polymerase. In preferred embodiments, the polymerase enzyme is a Phi29 polymerase, a Phi29-like polymerase, PolI polymerase or a BstI polymerase.

The present invention also provides methods of producing nucleic acid templates. Such methods generally comprise providing a nucleic acid molecule comprising a region of interest; digesting the nucleic acid molecule to provide a mixture comprising a fragment of the nucleic acid molecule comprising the region of interest and one or more additional fragments of the nucleic acid molecule that do not comprise the region of interest; ligating hairpin adapters to the ends of the fragment and the ends of the additional fragments; performing a second digestion of the additional fragments wherein the fragment comprising the region of interest is not cleaved, thereby converting the additional fragments into substrates for exonuclease activity; and subjecting the mixture to an exonuclease digestion, thereby digesting the additional fragments while not digesting the fragment comprising the region of interest, thereby synthesizing a nucleic acid template comprising the region of interest.

In certain aspects, methods for generating a single-stranded circular nucleic acid molecule are provided. In some embodiments, such a method comprises providing a double-stranded linear nucleic acid fragment; separating the fragment into two complementary strands in the presence of a single-stranded DNA binding protein, wherein the single-stranded DNA binding protein prevents reannealing of the strands; and treating the complementary strands with a ligase capable of ligating two ends of a single of the complementary strands together, thereby forming a single-stranded circular nucleic acid molecule. The separation can be performed using a helicase or heat-denaturation (e.g., wherein the single-stranded DNA binding protein is thermostable.) The separation and treating steps can be performed simultaneously or sequentially. In certain embodiments, the treating further comprises addition of an oligonucleotide complementary to both ends of a single complementary strand, wherein the oligo anneals to the ends and thereby positions them immediately adjacent to one another to facilitate the ligating.

In certain aspects, the invention provides methods for determining the sequence of an mRNA template, including the poly-A tail. In some embodiments, such methods comprise ligating a linker onto the 3′ end of a poly-A tail of an mRNA transcript; synthesizing a DNA complement to the mRNA transcript; degrading the mRNA transcript; generating a complement to the DNA complement to the mRNA transcript, thereby producing a double-stranded cDNA molecule; and sequencing the double-stranded cDNA molecule. In other embodiments, the methods comprise ligating a linker onto the 3′ end of a poly-A tail of an mRNA transcript; synthesizing a DNA complement to the mRNA transcript; degrading the mRNA transcript; generating a complement to the DNA complement to the mRNA transcript, thereby producing a double-stranded cDNA molecule; fragmenting the double-stranded cDNA molecule to produce cDNA fragments; selecting the cDNA fragments comprising a portion derived from the poly-A tail of the mRNA transcript; and sequencing the cDNA fragments so selected, wherein the sequence of the portion derived from the poly-A tail provides a length of the poly-A tail.

In yet further aspects, the invention provides methods for generating a cDNA sequencing template from a full-length mRNA transcript. For example, such methods comprise ligating a first linker onto the 3′ end of a poly-A tail; synthesizing a DNA complement to the mRNA transcript; degrading the mRNA transcript; and generating a complement to the DNA complement to the mRNA transcript, thereby producing a double-stranded cDNA molecule appropriate to serve as a template nucleic acid in a polymerase-mediated sequencing-by-synthesis reaction. Preferably, the full-length mRNA transcript is at least 100, 150, 200, 500, 1000, or 5000 base pairs in length. In certain embodiments, the first linker comprises a sequence complementary to a first primer used in the synthesizing of the DNA complement to the mRNA transcript, and can also comprise a poly-T region at its 3′ end. In come preferred embodiments, the first primer is biotinylated, and can be optionally used to select for the DNA complement to the mRNA transcript or the double-stranded cDNA molecule by binding to streptavidin, e.g., in chromatographic separations. In some embodiments, the methods further comprise selecting for cDNA comprising sequence complementary to the full-length mRNA transcript based upon the presence of sequence complementary to a 7 mG cap of the full-length mRNA transcript. In preferred embodiments, the generating comprises ligating a second linker to a 3′ end of the DNA complement to the mRNA transcript, wherein the second linker serves as a binding site for a primer, and wherein the primer serves as an initiation site for a primer extension reaction. In addition, the method can further comprise ligating the double-stranded cDNA molecule to two stem-loop adapters, thereby constructing a nucleic acid molecule having no free 3′ or 5′ ends.

In still further aspects, the invention provides methods for sequencing an mRNA transcript. In preferred embodiments, such methods comprise ligating a first linker onto the 3′ end of a poly-A tail region of a full-length mRNA transcript; synthesizing a DNA complement to the full-length mRNA transcript; degrading the full-length mRNA transcript; generating a complement to the DNA complement to the full-length mRNA transcript, thereby producing a double-stranded cDNA molecule; and ligating the double-stranded cDNA molecule to two stem-loop adapters to generate closed nucleic acid constructs having no free 3′ or 5′ ends; and sequencing the closed nucleic acid constructs. In preferred embodiments, the full-length mRNA transcript comprises both a poly-A tail region and a 7 mG cap, and is at least 100, 150, 200, 500, 1000, or 5000 base pairs in length. In certain embodiments, the first linker comprises a sequence complementary to a first primer used in the synthesizing of the DNA complement to the full-length mRNA transcript. Optionally the first primer can comprise a poly-T region at its 3′ end and/or is biotinylated. In some embodiments, the generating comprises ligating a second linker to a 3′ end of the DNA complement to the mRNA transcript, wherein the second linker serves as a binding site for a primer, and wherein the primer serves as an initiation site for a primer extension reaction. The methods can further comprise fragmenting the double-stranded cDNA molecule to produce cDNA fragments, and selecting the cDNA fragments comprising a portion comprising the poly-A tail region of the mRNA transcript. In certain preferred embodiments, the portion comprising the poly-A tail region of the mRNA transcript also comprises at least part of the 3′ untranslated region (3′UTR) of the mRNA transcript. The poly-A tail region can be at least 20, 30, 40, 50, 60, or greater than 70 nucleotides in length. Furthermore, sequencing of the closed nucleic acid constructs preferably provide sequence reads that encompass an entire sequence for the full-length mRNA transcript. Such sequencing can be performed iteratively such that the closed nucleic acid constructs are sequenced processively at least twice by a single polymerase enzyme, and is preferably performed using a single-molecule, real-time sequencing method, as described elsewhere herein.

### BRIEF DESCRIPTION OF THE DRAWINGS

FIG. 1 illustrates a preferred method for generating a SMRTbell™ template from an insert within a vector.

FIG. 2 illustrates a preferred method for generating a SMRTbell™ template having asymmetric adapters.

FIG. 3 illustrates a preferred method for generating double-stranded DNA fragments having a single-stranded region.

FIG. 4 illustrates preferred methods for generating double-stranded. DNA fragments having a single-stranded region.

FIG. 5 illustrates a preferred method for generating DNA templates using single mRNA transcripts.

FIG. 6 illustrates data generated during sequencing of a DNA template comprising cDNA generated from a full-length mRNA transcript.

FIG. 7 illustrates that data generated by sequencing a DNA template comprising a cDNA generated from a full-length mRNA transcript unambiguously identifies the mRNA transcript as OTUB1.

FIG. 8 illustrates data generated by sequencing templates comprising cDNA from various tissues and stages of Drosophila melanogaster.

FIG. 9 illustrates data generated by sequencing templates having poly-A sequences of different defined lengths: 20, 25, 30, 40, and 180 adenine bases.

FIG. 10 illustrates data generated by sequencing mRNA from the yeast RPS12 gene.

FIG. 11 illustrates data generated by sequencing templates comprising cDNA derived from β-actin mRNA

FIG. 12 illustrates the distribution of poly-A tail lengths of 3423 genes in S. cerevisiae measured using single-molecule real-time sequencing-by-synthesis.

FIG. 13 illustrates the distribution of untranslated region lengths of 3423 genes in S. cerevisiae measured using single-molecule real-time sequencing-by-synthesis.

FIG. 14 illustrates a region of a yeast genome containing a previously unidentified transcript.

FIG. 15 illustrates sequencing trace data from cDNA sequencing of a region of a yeast genome containing a previously unidentified transcript.

### DETAILED DESCRIPTION

Collecting reliable sequence data using high-throughput sequencing technologies depends in part on the availability of methods for the rapid and efficient production of high-quality nucleic acid templates. The present invention provides methods and compositions that can be useful in supplying templates to such high throughput DNA sequencing systems. The methods circumvent the need for costly, labor-intensive cloning and cell culture methods, which can limit sample production, e.g., preventing it from matching the capacities of modern sequencing systems (such systems are described in, e.g., Chan, et al. (2005) “Advances in Sequencing Technology” Mutation Research 573: 13-40; Levene et al. (2003) “Zero Mode Waveguides for Single Molecule Analysis at High Concentrations,” Science 299: 682-686; Korlach, et al. (2008) “Long, Processive Enzymatic DNA Synthesis Using 100% Dye-Labeled Terminal Phosphate-Linked Nucleotides” Nucleotides, Nucleosides, and Nucleic Acids 27:1072-1083; Travers, et al. (2010) “A flexible and efficient template format for circular consensus sequencing and SNP detection” Nucl. Acids Res. 38(15):e159; Korlach, et al. (2010) “Real-time DNA sequencing from single polymerase molecules” Methods in Enzymology 472:431-455; and Eid et al. (2009) Science 323:133-138, the disclosures of which are incorporated herein by reference in their entireties for all purposes). Also, in certain embodiments they allow for primer-free template-directed nascent strand synthesis, e.g., from an amplified template. Accordingly, a reduction in sequencing costs, at least with regards to the cost of primers for initiation of nascent strand synthesis, is a benefit of the improved methods provided herein. The methods can be scaled to accommodate template production for a variety of sequencing applications, e.g., sequencing individuals' genomes, gene expression profiling (Spinella, et al. (1999) “Tandem arrayed ligation of expressed sequence tags (TALEST): a new method for generating global gene expression profiles.” Nucleic Acids Res 27: e22; Veleulescu, et al. (1995) “Serial analysis of gene expression.” Science 270: 484-487); and others.

The nucleic acids to be sequenced can be obtained from any source of interest, and can comprise DNA, RNA, and mimetics, analogs, and derivatives thereof. They can be isolated from cells, cell cultures, tissue samples, bodily fluids, viral samples, genomic nucleic acid samples, cDNA preparations, environmental samples, forensic samples, or synthetic sources. Nucleic acids can be cloned, amplified, transcribed, ligated, fragmented, or otherwise manipulated according to standard methods to provide the nucleic acid to be sequenced as these manipulations do not render the nucleic acid unsuitable for subsequent sequencing as described herein. It will be understood that such nucleic acids may comprise modified, non-canonical, and/or non-natural nucleotides or nucleotide analogs, many of which are described in U.S. patent application Ser. No. 12/945,767, filed Nov. 12, 2010, which is incorporated herein by reference in its entirety for all purposes.

While nucleic acids can be cloned prior to preparation according to the present invention, in many cases cloning will not be necessary. In single-molecule sequencing applications, large quantities of nucleic acids are not needed to provide a nucleic acid of interest. Instead, genomic DNA or other nucleic acids can be sequenced directly without an intermediate cloning step. Alternatively, and in certain preferred embodiments, the nucleic acids can be amplified prior to cloning for one or more amplification cycles. Appropriate amplification methods can include PCR, linear PCR (linear rather than exponential amplification), RT-PCR, RACE (rapid amplification of cDNA ends), LCR, transcription, strand displacement amplification (SDA), multiple-displacement amplification (MDA), rolling circle replication (RCR), those described in U.S. Patent Publication No. 20100081143 (incorporated herein by reference in its entirety for all purposes), or other methods known to those of ordinary skill in the art.

Procedures for isolating, cloning, and amplifying nucleic acids are replete in the literature and can be used in the present invention to provide a nucleic acid to be sequenced. Further details regarding nucleic acid cloning, amplification and isolation can be found in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”); The Nucleic Acid Protocols Handbook Ralph Rapley (ed) (2000) Cold Spring Harbor, Humana Press Inc (Rapley); Current Protocols in Molecular Biology, F. M. Ausubel et al., eds., Current Protocols, a joint venture between Greene Publishing Associates, Inc. and John Wiley & Sons, Inc., (supplemented through 2007) (“Ausubel”)); PCR Protocols A Guide to Methods and Applications (Innis et al. eds) Academic Press Inc. San Diego, Calif. (1990) (Innis); Chen et al. (ed) PCR Cloning Protocols, Second Edition (Methods in Molecular Biology, volume 192) Humana Press; in Viljoen et al. (2005) Molecular Diagnostic PCR Handbook Springer, ISBN 1402034032; Demidov and Braude (eds) (2005) DNA Amplification: Current Technologies and Applications. Horizon Bioscience, Wymondham, UK; and Bakht et al. (2005) “Ligation-mediated rolling-circle amplification-based approaches to single nucleotide polymorphism detection” Expert Review of Molecular Diagnostics, 5(1) 111-116. Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

A plethora of kits are commercially available for the purification of plasmids or other relevant nucleic acids from cells, (see, e.g., EasyPrep™, FlexiPrep™, both from Pharmacia Biotech; StrataClean™, from Stratagene; QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. See Sambrook, Ausubel and Berger. In addition, essentially any nucleic acid can be custom or standard ordered from any of a variety of commercial sources, such as Operon Technologies Inc. (Huntsville, Ala.).

### Preparing Genomic DNA

As described above, the single-stranded nucleic acids, e.g., linear or circular nucleic acids, that are provided by the methods described herein, e.g., for use in single molecule sequencing reactions, can be derived from a genomic DNA. Genomic DNA can be prepared from any source by three steps: cell lysis, deproteinization and recovery of DNA. These steps are adapted to the demands of the application, the requested yield, purity and molecular weight of the DNA, and the amount and history of the source. Further details regarding the isolation of genomic DNA can be found in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2008 (“Sambrook”); Current Protocols in Molecular Biology, F. M. Ausubel et al., eds., Current Protocols, a joint venture between Greene Publishing Associates, Inc. and John Wiley & Sons, Inc (“Ausubel”); Kaufman et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley). In addition, many kits are commercially available for the purification of genomic DNA from cells, including Wizard™ Genomic DNA Purification Kit, available from Promega; Aqua Pure™ Genomic DNA Isolation Kit, available from BioRad; Easy-DNA™ Kit, available from Invitrogen; and DnEasy™ Tissue Kit, which is available from Qiagen.

Preparing cDNA

The template nucleic acids that can be prepared by the methods described herein, e.g., for use with high-throughput sequencing systems can also be derived from a cDNA, e.g. cDNAs prepared from mRNA obtained from, e.g., a eukaryotic subject or a specific tissue derived from a eukaryotic subject. Data obtained from sequencing the nucleic acid templates derived from a cDNA library, e.g., using a high-throughput sequencing system, can be useful in identifying, e.g., novel splice variants of a gene of interest or in comparing the differential expression of, e.g., splice isoforms of a gene of interest, e.g., between different tissue types, between different treatments to the same tissue type or between different developmental stages of the same tissue type.

mRNA can typically be isolated from almost any source using protocols and methods described in, e.g., Sambrook and Ausubel. The yield and quality of the isolated mRNA can depend on, e.g., how a tissue is stored prior to RNA extraction, the means by which the tissue is disrupted during RNA extraction, or on the type of tissue from which the RNA is extracted. RNA isolation protocols can be optimized accordingly. Many mRNA isolation kits are commercially available, e.g., the mRNA-ONLY™ Prokaryotic mRNA Isolation Kit and the mRNA-ONLY™ Eukaryotic mRNA Isolation Kit (Epicentre Biotechnologies), the FastTrack 2.0 mRNA Isolation Kit (Invitrogen), and the Easy-mRNA Kit (BioChain). In addition, mRNA from various sources, e.g., bovine, mouse, and human, and tissues, e.g. brain, blood, and heart, is commercially available from, e.g., BioChain (Hayward, Calif.), Ambion (Austin, Tex.), and Clontech (Mountain View, Calif.).

Once the purified mRNA is recovered, reverse transcriptase is used to generate eDNAs from the mRNA templates. Methods and protocols for the production of cDNA from mRNAs, e.g., harvested from prokaryotes as well as eukaryotes, are elaborated in cDNA Library Protocols, I. G. Cowell, et al., eds., Humana Press, New Jersey, 1997, Sambrook and Ausubel. In addition, many kits are commercially available for the preparation of cDNA, including the Cells-to-cDNA™ II Kit (Ambion), the RETROscript™ Kit (Ambion), the CloneMiner™ cDNA Library Construction Kit (Invitrogen), and the Universal RiboClone cDNA Synthesis System (Promega). Many companies, e.g., Agencourt Bioscience and Clontech, offer cDNA synthesis services.

### Cleaving Nucleic Acids to Produce Fragments

In some embodiments of the invention described herein, nucleic acid fragments are generated from nucleic acid sample, e.g., a genomic DNA or a cDNA sample. There exist a plethora of ways of generating nucleic acid fragments from a genomic DNA, a cDNA, or a DNA concatamer. These include, but are not limited to, mechanical methods, such as sonication, mechanical shearing, nebulization, hydroshearing, and the like; enzymatic methods, such as exonuclease digestion, restriction endonuclease digestion, and the like; and electrochemical cleavage. These methods are further explicated in Sambrook (Molecular Cloning: A Laboratory Manual. New York: Cold Spring Harbor Laboratory Press; 1989) and Ausubel (Current Protocols in Molecular Biology. New York: John Wily; 2001), which are incorporated herein by reference in their entireties for all purposes.

### Amplifying Nucleic Acid Fragments

In certain embodiments described herein, amplification of the sample nucleic acid is performed. The most widely used in vitro technique for amplifying nucleic acids is the polymerase chain reaction (PCR), which requires the addition of a template of interest, e.g., a DNA comprising the sequence that is to be amplified, nucleotides, oligonucleotide primers, buffer, and an appropriate polymerase to an amplification reaction mix. In PCR, the primers anneal to complementary sequences on denatured template DNA and are extended with a thermostable DNA polymerase to copy the sequence of interest. As a result, nucleic acids comprising sequence complementary to a template strand to which a primer was bound are synthesized, and these nucleic acids comprise the primer used to initiate the polymerization reaction. Repeated cycles of PCR generate many copies of the template strand and its complement. Primers ideally comprise sequences that are complementary to the template. However, they can also comprise sequences having non-complementary, non-canonical, and/or modified nucleotides or sequences including, but not limited to, restriction sites, cis regulatory sites, oligonucleotide hybridization sites, protein binding sites, DNA promoters, RNA promoters, sample or library identification sequences, combinations of deoxyribonucleotides and ribonucleotides, and the like. Primers can comprise modified nucleotides, such as methylated, biotinylated, or fluorinated nucleotides; and nucleotide analogs, such as dye-labeled nucleotides, non-hydrolysable nucleotides, and nucleotides comprising heavy atoms. Primers comprising such modifications can be custom synthesized, and PCR can be a useful means by which to integrate the modifications into nucleic acids. Specific methods that use primers having modifications are further described below. As noted above, modified, non-canonical, and/or non-natural nucleotides or nucleotide analogs are described in U.S. patent application Ser. No. 12/945,767, filed Nov. 12, 2010, and incorporated herein by reference in its entirety for all purposes. For example, in certain embodiments inclusion of a modification alters the efficiency of hybridization between the primer and the primer binding site and/or creates a recognition site for a further modification of the primer or resulting amplicons, e.g., by an enzyme such as a glycosylase or nuclease. In specific embodiments, ribo- or deoxyribonucleotides within a primer sequence comprise 2′ O-methyl-modified sugar groups, and these modified nucleotides increases the melting temperature and the kinetics of hybridization, thereby promoting annealing to the primer binding site and enhancing the stability of the hybridized complex at a wider range of temperatures. (See, e.g., Majlessi, et al. (1998) Nucl. Acids Res. 26(9): 224-229, incorporated herein by reference in its entirety for all purposes.) In addition, 2′ O-methyl-modified nucleotides are less susceptible to a variety of ribo- and deoxyribonucleases. In certain preferred embodiments, the number of 2′ O-methyl-modified nucleotides within a primer is at least about 6, 7, 8, 9, or 10. The modified nucleotides may be adjacent to one another, or spaced apart, and can be located internally or terminally within the primer.

Primers are useful not only for amplification of nucleic acids, but also for other functions. For example, binding of a primer to a template can provide a binding an initiation site for polymerase-mediated nascent strand synthesis. Such primers comprise sequence complementary to the template, and can optionally comprise non-complementary, non-canonical, and/or modified nucleotides or sequences, as described above for primers used in nucleic acid amplification. For example, use of 2′ O-methyl-modified nucleotides, which have a lower melting temperature and are therefore more stable, can enhance the proportion of templates that are bound by a polymerase enzyme. Other modifications that could be used include locked nucleic acids and peptide nucleic acids. Likewise, many methods and compositions described herein include the use of other oligonucleotides, such as splint oligonucleotides or adapters. Like primers, adapters can also comprise sequence complementary to a sample nucleic acid (e.g., sticky ends), and non-complementary, non-canonical, and/or modified nucleotides or sequences. Specific embodiments of such oligonucleotides (e.g., primers, splints, adapters) are described in detail elsewhere herein.

### Methods for Generating Linear and Circular Sequencing Templates

The invention provides methods and compositions for generating a population of nucleic acid templates appropriate for template-directed nascent strand synthesis, e.g., catalyzed by a polymerase enzyme. In preferred embodiments, the templates are useful for single-molecule, real-time nucleic acid sequencing, e.g. SMRT® sequencing from Pacific Biosciences (Menlo Park, Calif.).

Nucleic acid templates appropriate for sequencing can be linear or circular, and can be single- or double-stranded. Where single-stranded template is desired, a double-stranded fragment can be heat-denatured, unwound using a helicase enzyme, or one strand of the double-stranded fragment can be selectively degraded, e.g., with an exonuclease. Circular or linear single-stranded templates can optionally be stabilized by addition of a single-stranded DNA binding protein. Such templates can comprise deoxyribonucleotides, ribonucleotides, and/or analogs, mimetics, or modifications thereof. Nucleotide modifications that can be present in nucleic acid templates include those described in detail in International Application No. PCT/US2011/060,338, filed Nov. 11, 2011, the disclosure of which is incorporated herein by reference in its entirety for all purposes. Typically, double-stranded linear templates are generated by cleaving larger nucleic acid molecules, and, optionally amplifying the resulting fragments. Alternatively, linear fragments can also be generated by amplifying from an initial, unfragmented, nucleic acid sample. For example, if a particular region of a genome is to be sequenced, PCR primers specific for that region can be used to amplify the region of interest, thereby generating linear nucleic acids from that region. Optionally, the ends of such fragments are modified, e.g., by adding oligonucleotide adapters, treating with nucleases, and/or adding polynucleotide tails. Specific embodiments are further described in detail elsewhere herein.

Where circular template is desired, e.g., for generation of redundant sequence information as described in U.S. Pat. Nos. 7,476,503, 7,906,284, and 7,901,889, (all incorporated herein by reference in their entireties for all purposes), various methods for conversion of linear nucleic acid to circular nucleic acid can be performed, e.g., ligation of the ends of a double-stranded linear molecule to adapters (e.g., blunt adapters) that are subsequently ligated together to form a circular double-stranded molecule, e.g., using T4 or Taq ligase. Where the nucleic acid fragments were generated using a method that produces random overhangs, adapters having random overhangs can be used. Alternatively, the ends of the fragments can be subjected to a single-strand-specific exonuclease to “repair” them, creating blunt ends to which adapters (e.g., universal adapters) can be ligated. In some embodiments, dATPs can be added to the ends of a linear, double-stranded fragment, e.g., using terminal transferase. The resulting poly-A sequences serve as binding sites for poly-T overhangs at both ends of an adapter. Once annealed, the adapter brings the ends of the double-stranded fragment in close proximity with one another. Any gaps in the annealed complex can be filled in using an appropriate polymerase enzyme, and a double-strand-specific ligase is used to connect the sugar-phosphate backbones of the fragment to the adapter. In some embodiments, the Cre-Lox recombination system can be used to generate circular nucleic acid templates, where adapters annealed to the ends of the fragments comprise loxP sites that can undergo recombination catalyzed by the Cre recombinase to generate a circular nucleic acid. More details of this method are provided in, e.g., Araki, et al. (1987) J. Biochem (Tokyo) 122:977-82; and Nagi, A. (2000) Genesis 26:99-109, the teachings of which are incorporated herein by reference in their entirety for all purposes. In certain cases, the mitochondrial transcription factor TFAM can be added to a ligation reaction to help condense the nucleic acids, thereby increasing the effective concentration of the ends and facilitating ligation of single- or double-stranded nucleic acids. (See, e.g., Kaufman, et al. (2007) Mol. Biol. Cell. 18(9):3225-3236.)

For single-stranded circular templates, a double-stranded circular template can be treated to remove one strand, e.g., by specific nicking and degrading, heat-denaturation, helicase treatment, and the like. For example, adapters used to create a double-stranded, circular nucleic acid can comprise nicking sites or other modifications that can target base removal and/or phosphodiester backbone cleavage on only one strand, which can then be removed by enzymatic (e.g., helicase or nuclease), chemical, or thermal means to produce a circular, single-stranded nucleic acid molecule. In certain specific embodiments, an adapter used to circularize a linear, double-stranded nucleic acid comprises dUTPs on one of its two strands. Alternative embodiments comprise the use of two adapters, e.g., where the adapters comprise sticky ends that are complementary to allow hybridization and, thereby, circularization. The sticky end of one adapter can comprise dUTPs while the sticky end of the other does not such that when hybridized together only a single strand of the resulting circular molecule comprises the dUTPs. Although some of the linear, double-stranded molecules will be ligated to the same adapter at both ends, these will not form circles and can therefore be degraded by nuclease treatment to remove them from the mixture. Further, two different adapters can be used, each of which is specific to only one of the ends of the linear, double-stranded fragment to produce a fragment with a different adapter at each end.

After ligation, the resulting circular nucleic acid having the UTPs on one strand is treated with (a) uracil DNA glycosylase, which hydrolyzes the N-glycosylic bond, flipping out the uracil base, and (b) apurinic (AP) endonuclease (e.g., endonuclease IV), which cleaves the phosphodiester backbone leaving a one-base gap. The damaged strand is then removed as described above to produce a circular, single-stranded nucleic acid molecule. Other glycosylases can also be used to create an apurinic or apyrimidinic site that can be acted upon by an AP endonuclease, and each is specific for a particular type of modification, e.g., methylated bases, oxidized bases, and the like, each of which is a candidate for inclusion in an adapter for circularizing a double-stranded, linear nucleic acid as long as the same modification is not present in the original linear double-stranded molecule to be circularized. (For a review of glycosylases and base excision repair, see Krokan, et al. (1997) Biochem. J. 325:1-16, the disclosure of which is incorporated herein by reference in its entirety for all purposes.) Alternatively, an adapter can be constructed that already contains an abasic site (rather than a modification to be converted to an abasic site.) Yet further, the adapter can comprise one or more nicks or gaps, which allow removal of the “damaged” strand in a subsequent step. For example, a gap in the adapter can serve as an entry point for a helicase or exonuclease that would remove or degrade the gapped strand. A nicked or gapped adapter would also permit thermal removal of the nicked or gapped strand after ligation. In still further embodiments, the 5′-end of one of the adapters lacks a phosphate group (or the 3′ end of one of the adapters lacks a hydroxyl), resulting in ligation of one strand and an unligated nick in the strand lacking the phosphate (or hydroxyl) group. Where a gap is preferred over a nick, a limited exonuclease can be used to degrade the nicked strand to produce a gap. The limited exonuclease can be performed in various ways, including limiting the time of the reaction, using reaction conditions that tightly control the nuclease activity, or by including in the adapter modifications that block further degradation, e.g., phosphorothioate modifications. (See, e.g., Liu, et al. (2010) Protein Science 19:967-973, incorporated herein by reference in its entirety for all purposes.) In certain preferred embodiments, a circular nucleic acid with a gapped strand is used directly in a sequencing reaction, where the gap is the polymerase binding and initiation site and the gapped strand is removed by strand displacement catalyzed by the polymerase enzyme.

Alternatively, kits are available for preparation of single-stranded circles, e.g., using CircLigase™ ssDNA ligase (Epicenter®, an Illumine® company; Madison, Wis.). Two caveats with CireLigase™, however, are that it (1) displays sequence bias, so is not appropriate for all nucleic acids, and (2) has a reaction temperature of only 60° C., so is below the melting temperature of nucleic acids of about 100 bp. The result of the latter is inefficient circular ssDNA production due to the reannealing of an initial dsDNA fragment, preventing efficient ssDNA ligation. In certain embodiments, ligation reactions include one or more proteins that separate dsDNA and/or prevent re-annealing (e.g., helicase or single-stranded DNA binding protein (SSB)) and are preferably thermostable (e.g., from New England Biolabs®, Ipswich, Mass.; and/or Biohelix™, Beverly, Mass.). Such proteins have previously been used to enhance isothermal DNA amplifications (Vincent, et al. (2004) EMBO Reports 5(8):795-800, incorporated herein by reference in its entirety for all purposes). In particular embodiments, a ligase reaction is heated to denature the dsDNA in the presence of a thermostable SSB protein, which inhibits or prevents reannealing of the resulting single strands. The temperature is subsequently lowered to the proper temperature for the CircLigase™ reaction, and ligation is performed. Alternatively, a helicase enzyme can be used to separate the strands in the presence of an SSB protein, which prevents their reannealing prior to ligation. Optionally, adapters having a specific primer binding site can be ligated to both ends of the initial dsDNA fragment, and primers complementary to the primer binding sites included in the ligation mixture. Annealing of the primers to the primer binding sites could displace any SSB protein, thereby potentially ameliorating any interference bound SSB protein might have with the ligase reaction. Further, if the primers extended to the end of the single-stranded fragment, a double-strand-specific ligase can be used to perform the ligation reaction; in certain embodiments, the primers are also ligated to create a double-stranded region on the resulting circular single-stranded molecule. This double-stranded region can optionally be used as an initiation site for a polymerase during a subsequent template-directed synthesis reaction, or the ligated primer can be removed, e.g., by heating, helicase activity, etc.

Other methods for generating single-stranded circular template nucleic acids include using a “splint” oligonucleotide that is complementary to both ends of a linear, single-stranded fragment. The splint oligonucleotide brings the ends of the single-stranded fragment together so they can be ligated using a double-strand-specific ligase (e.g., T4 or Tag ligase) to generate a single-stranded, circular molecule. (It will be understood that where annealing of a splint oligo to the ends of a single-stranded nucleic acid results in a gap between the 3′- and 5′-termini, a gap-filling operation is carried out using an appropriate polymerase enzyme (e.g., T4 DNA polymerase) prior to the ligation step, and such gap-filling methods are known to those of ordinary skill in the art) Where the linear fragment has defined ends, e.g., as would be generated from restriction digestion of the original sample nucleic acids, the splint oligonucleotide is designed to be complementary to the ends. Alternatively, where the sequence of the ends is unknown, adapters are annealed to both ends of the single-stranded fragment, and the splint oligonucleotide is complementary to the annealed adapters. In yet further embodiments, a single-stranded circular template can be generated from a double-stranded linear template using a combination of a splint oligonucleotide and double-stranded adapters having functionally single-stranded “split ends” comprising non-complementary sequences. In such embodiments, the same adapter is ligated to both ends of the linear, double-stranded nucleic acid, and the product is a double-stranded molecule in which the two termini are in a single-stranded form, having no complement with which to bind. This double-stranded molecule is subsequently rendered single-stranded (e.g., using heat-denaturation, chemical-denaturation, heliease activity, etc.), and because the split ends have non-complementary sequences, the 5′- and 3′-ends of the resulting single strands will not anneal together. A splint oligo having sequence complementary to both of the strands of the split ends is present in or added to the mixture, and annealing of the splint oligo to the 3′- and 5′-ends of one of the single-stranded molecules brings the ends together, facilitating ligation and thereby circular, single-stranded nucleic acid formation. In some embodiments, a splint oligo used to circularize a single-stranded nucleic acid can subsequently be used as a primer for polymerase initiation of sequencing by synthesis on that single-stranded nucleic acid, or it can be removed prior to priming and strand synthesis. Similarly, where adapters are added to the ends of the single-stranded fragments, a double-stranded splint having sticky ends complementary to the adapters can be used in combination with a double-strand-specific ligase to create a single-stranded circle with a short double-stranded region that can be used to prime the subsequent polymerase reaction. In preferred embodiments, the splint oligonucleotide is 20-40 nucleotides in length. Further, the yield of the ligation can be increased by first subjecting the linear, single-stranded fragments to a denaturing step at 95° C., which eliminates any possible secondary structure. The annealing of the splint and subsequent ligation are performed at 60° C. using a thermostable dsDNA ligase, e.g., Taq ligase. The process is repeated dozens of times, e.g., at least about 40, 50, 60, 70, or 80 times to increase the total yield. Methods of ligating single-stranded nucleic acids hybridized to a complementary nucleic acid are provided, e.g., in Nilsson, et al. (1994) Science 265:2085-2088, which is incorporated herein by reference in its entirety for all purposes.

In preferred embodiments, circular, a single-stranded template is constructed from a linear, double-stranded fragment such that the resulting circular construct comprises both strands of the double-stranded fragment in a single contiguous strand. For example, a hairpin adapter can be added to each end of the double-stranded fragment; separation of the strands results in a closed, circular molecule having both strands of the original double-stranded fragment separated by the regions corresponding to the adapters. Such templates are termed “SMRTbell™ templates” herein, and such templates and derivations thereof are described in detail in Travers, et al. (2010) Nucl. Acids Res. 38(15):e159; and U.S. patent application Ser. Nos. 12/413,258, filed Mar. 27, 2009; 13/019,220, filed Feb. 1, 2011; and 12/982,029, filed Dec. 30, 2010, the disclosures of all of which are incorporated herein by reference in their entireties for all purposes. One important benefit of using SMRTbell™ templates during sequencing-by-synthesis reactions is the ability to generate redundant sequence information, both as a result of generating sequence information for both strands of the original double-stranded nucleic acid, but also by repeatedly or iteratively sequencing the entire template. For example, a single polymerase enzyme with strand displacement activity can initiate at a single position (e.g., at a primer) and synthesize a nascent strand that is complementary to the template; after passing around the template one time, the polymerase can continue around repeatedly, displacing the nascent strand from the template in front of it, to produce a long, concatemeric nascent strand comprising multiple complementary copies of the template. By monitoring nucleotide incorporation into the concatemeric nascent strand, multiple sequence reads are generated for both strands of the original double-stranded fragment. The adapter sequences used to construct SMRTbell™ templates preferably comprise specialized sequences, such as primer binding sites, regions of internal complementarity to provide a short, double-stranded “stem” region that forms a double-stranded terminus appropriate for ligation to the end of a double-stranded nucleic acid fragment. The portion of the SMRTbell™ template adapter that is not within the stem region is sometimes referred to as the “single-stranded portion” or the “loop” in a stem-loop adapter. SMRTbell™ template adapters may also comprise sequences that regulate polymerase activity (e.g., causing the polymerase to pause or stop). SMRTbell™ template adapters typically comprise canonical nucleotides, but can also comprise non-canonical or modified bases, such as those described in U.S. patent application Ser. No. 12/945,767, filed Nov. 12, 2010. For example, in some embodiments one or more nucleotides having a 2′ O-methyl-modified sugar group are included in the adapter sequence. Similar to including these modified nucleotides in primer sequences as described supra, inclusion of these modified nucleotides in an adapter sequence within a primer binding site increases both the melting temperature and kinetics of primer binding, thereby enhancing stabilization of the template-primer complex. An additional feature beneficial to certain embodiments is that the presence of 2′ O-methyl-modified nucleotides in the template sequence is inhibitory for polymerase synthesis, and can block progression of the enzyme. (See, e.g., Stump, et al. (1999) Nucl. Acids Res. 27(23):4642-4648, which is incorporated herein by reference in its entirety for all purposes.) In practice, several consecutive 2′ O-methyl-modified nucleotides in the single-stranded portion of the SMRTbell™ template adapter provide efficient cessation of nascent strand synthesis, and in preferred embodiments the number of consecutive 2′ O-methyl-modified nucleotides is at least about 6, 7, 8, 9, or 10. In alternative embodiments, the adapter comprises deoxyuracils and is treated with uracil deglycosylase to create abasic sites that also serve to terminate polymerization. (See, e.g., U.S. Ser. No. 12/982,029, filed Dec. 30, 2010.) Other modified bases can also be used to terminate polymerization, e.g., locked nucleic acids, 2′-fluoro-modified nucleotides, and the like. This feature is useful where one wishes to only sequence a single strand of the original, double-stranded fragment. Since often a SMRTbell™ template has the same adapter at both ends of the double-stranded fragment, a polymerase binding at a primer bound to one adapter (at a position over or downstream of the 2′ O-methyl-modified nucleotides) will initiate synthesis and process a first strand, but will terminate synthesis at the 2′ O-methyl-modified nucleotides within the second adapter sequence.

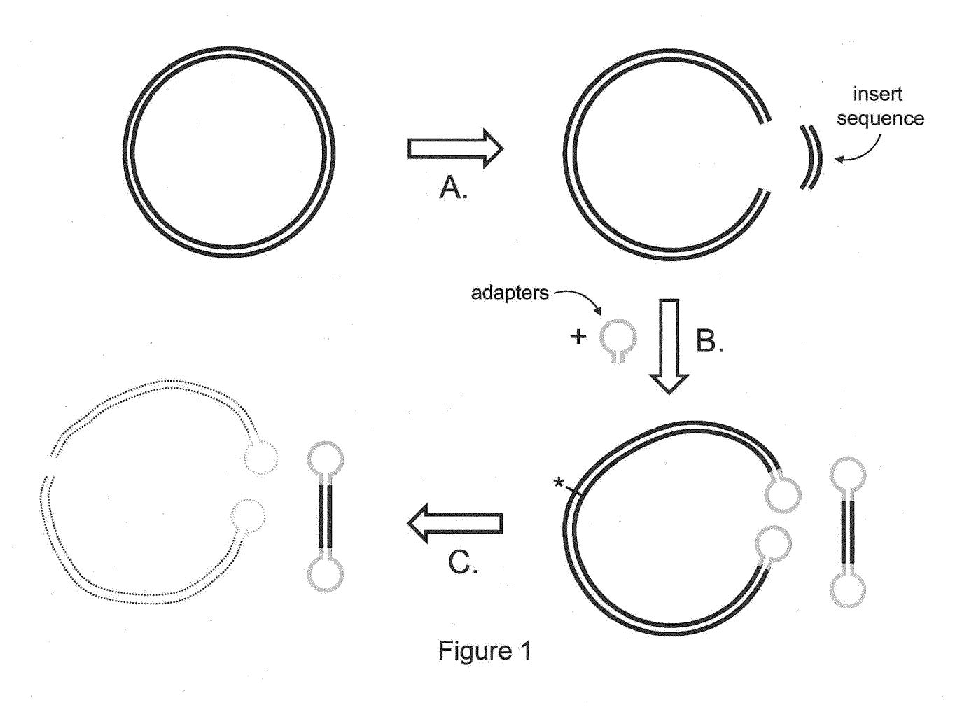

In some embodiments, it is desirable to sequence an insert within a vector (e.g., from a cloning library). A challenge in the generation of SMRTbell™ templates comprising inserts is how to remove the vector from the preparation so that only the insert is present in the final SMRTbell™ templates. Traditional cleavage of the insert from the vector and gel purification methods cause damage to the nucleic acids (e.g., from exposure to intercalation dyes and UV light) and the yield of recovered insert can be too low for efficient ligation to SMRTbell™ template adapters. Certain aspects of the present invention provide methods for efficiently creating SMRTbell™ templates from inserts without the damage and low yields afforded by the traditional methods, and an exemplary embodiment is depicted in FIG. 1. In preferred embodiments, an insert is removed from a vector, e.g. using known restriction sites, e.g., in step A of FIG. 1. SMRTbell™ template adapters are added in step B. At this point, SMRTbell™ templates are formed from both the insert sequences and the vector sequences.

Subsequently, as shown in step C, the mixture is treated with a restriction endonuclease that has a recognition site (*) within the vector sequence, but not within the insert or adapter sequences. Restriction sites unlikely to be present in the insert sequence can be chosen in various ways, e.g., by selecting those recognized by rare-cutting restriction enzymes, or by using knowledge of the insert sequence, e.g., where it has been previously sequenced. Further, restriction enzymes can be used that are sensitive to modifications in the canonical cleavage site. For example, the insert can be subjected to a modification (e.g., methylation, hydroxylation, etc.) that alters such restriction sites so they are unrecognizable by the enzyme. Alternatively and preferably, the vector can be treated to introduce modifications that are specifically cleaved by enzymes but that do not occur within the insert sequence. For example, the specific type of modification may not exist naturally in an organism from which the insert was isolated or derived. For example, certain types of methylated bases are found in bacterial species but not human DNA, so an insert derived from human DNA would be known to lack such methylated bases. A combination of the appropriate glycosylase and endonuclease would create a nick in the vector, and in doing so an entry point for a nuclease. Finally, the mixture is treated with one or more exonucleases (e.g., ExoIII, ExoVII). The cleaved vector will be degraded, leaving only the insert-containing SMRTbell™ templates, which were not cut by the endonuclease and are therefore protected from the exonuclease(s). The degradation step can be performed simultaneously with the cleavage of the vector, as shown in FIG. 1, or these steps can be performed sequentially. The insert-containing SMRTbell™ templates can then be used in various analyses, e.g., in sequencing-by-synthesis reactions, or optionally, the inserts can be removed from the SMRTbell™ templates for further manipulations. In some embodiments, the SMRTbell™ adapters comprise additional useful sequences, e.g., additional restriction enzyme recognition sequences (e.g., in the stem portion), binding sites for nucleic acid binding proteins (e.g., for use in further purification steps such as affinity chromatography or chromatin immunoprecipitation).

In some embodiments, it is preferable to have a different SMRTbell™ template adapter ligated to each end of a double-stranded fragment. Many protocols for constructing SMRTbell™ templates produce templates having symmetric stem-loop adapters. That is, the same SMRTbell™ stem-loop adapter is annealed at both ends of a double-stranded nucleic acid fragment. In such embodiments, both ends of the resulting template have primer binding sites for the polymerase, as well as any other moiety desired (e.g., stop or pause sites, registration sequences, recognition sequences for nucleic acid-modifying or binding proteins/enzymes, etc.) In contrast, SMRTbell™ templates having asymmetric stem-loop adapters allow the practitioner to choose different characteristics at each end of the template, and potentially could provide more flexibility and/or better control of a subsequent analytical reaction, e.g., sequencing reaction. Similar to the method above, the method involves use of a vector-insert construct, which can be derived from a library, or can be constructed specifically for the construction of an asymmetric SMRTbell™ template, e.g., by inserting a double-stranded fragment into a vector of known sequence by standard molecular biology methods. The method involves cleavage of the insert-vector construct by three restriction enzymes, and this digest can be performed sequentially or simultaneously, where the restriction enzymes efficiently operate under the same or substantially similar reaction conditions. Preferably, recognition sequences for the restriction enzymes are not present in the insert. As described above, various strategies can be used to decrease the chance that a recognition site is found within the insert, including but not limited to the use of rare cutting endonucleases and the use of modification-specific endonucleases (where the modification is absent from the insert). Two of the restriction enzymes cleave the vector at locations that flank the insert site, and the third restriction enzyme cleaves the vector at a location further from the insert site. Two different stem-loop adapters are added, each with different sticky ends such that each of the cleavage sites that flank the insert site will anneal to a different adapter. No adapter is specific for the cleavage site that is distal from the insert site. A ligation reaction is performed to ligate the adapters to the cleavage sites nearest the insert site. At this point, a SMRTbell™ template asymmetric adapters that comprises the insert sequence has been formed, and two constructs having a single adapter at one end and cleaved restriction site at the other are also present in the mixture. Treatment with one or more exonucleases degrades the single-adapter constructs, but the SMRTbell™ template is protected since it has no double-strand or single-strand termini accessible to the exonuclease activity. In this way, an asymmetric SMRTbell™ template is generated.

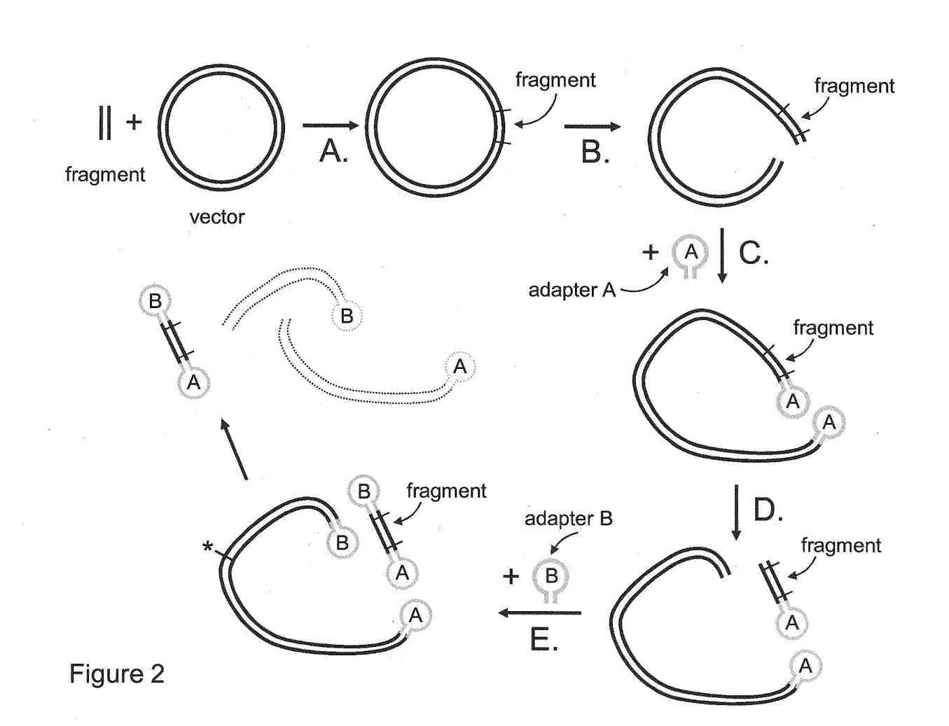

FIG. 2 provides an illustrative embodiment of a step-by-step preparation of an asymmetric SMRTbell™ template starting from a double-stranded linear fragment, which could be, e.g., a product of a restriction digest, amplification reaction, cDNA synthesis, fragmentation, etc. First, the double-stranded linear fragment (insert) is ligated into a vector having a first restriction site upstream of the insert site and a second, different, restriction site downstream of the insert site. Where the termini of the double-stranded fragment are unknown, they can be further processed to allow insertion into the vector, e.g., by creation of blunt ends, addition of adapters having sticky ends that hybridize with sticky ends at the insert site of the vector, and the like. For example, terminal transferase can be used to add a tail of a first type of nucleotide to the insert and a tail of a second type of nucleotide to the vector (subsequent to opening the insert site). The first type of nucleotide is complementary to the second type of nucleotide, so the tailing would produce complementary overhangs between the insert and vector. This would prevent insert-insert and vector-vector complexes, but there could still be insert-vector-insert-vector complexes (and multiples thereof) formed. The frequency of these can be reduced by adjusting the ratio of insert to vector in the ligation reaction, as is conventionally done for cloning applications. One of the restriction sites flanking the insert site is cleaved to open the vector-insert construct. A first stem-loop adapter (A) having a double-stranded terminus complementary to the overhang at the ends of the open construct is ligated thereto, resulting in a “long SMRTbell™ template” comprising both the vector and insert sequences. Next, the other restriction site flanking the insert site is cleaved to separate the construct into two portions: one having the insert (plus a small amount of vector sequence) and one having the majority of the vector sequence and no insert. A second stem-loop adapter (B) having a double-stranded terminus complementary to the overhang at the double-stranded ends of the two portions is ligated thereto, resulting in two SMRTbell™ templates: one having the insert (plus a small amount of vector sequence) and one having the majority of the vector sequence and no insert. While not required, this staged approach to removing the insert from the vector allows temporally separate ligations of stem-loops at each end of a fragment, therefore further ensuring attachment of a different stem-loop adapter at each end. The vector sequence present in the “vector-only” SMRTbell™ template also comprises a third restriction site that is not present, or is highly unlikely to be present, in the insert nucleic acid (e.g., a NotI restriction site). This template is subsequently cleaved with an appropriate restriction enzyme and is further degraded with exonuclease enzymes (e.g., ExIII & ExoVII). The insert-containing SMRTbell™ template does not have a double-stranded nucleic acid end, and so will not be susceptible to the exonuclease treatment.

A single-stranded nucleic acid could be used as an insert in the above-described method if it were rendered double-stranded by synthesis of the complementary strand. This could be done before insertion into the vector, or double-stranded adapters could be added to the ends to allow ligation of the fragment into the vector prior to treatment with a polymerase, which would synthesize the complementary strand. If a nucleic acid of interest is already present in a vector, e.g., a cloning vector from a genomic library, then the insertion step is omitted as long as the vector already has the necessary elements for the other steps in the method, e.g., appropriate restriction sites. The ratio of insert to vector present in the initial ligation reaction to produce the insert-vector construct can be optimized to increase the likelihood that only a single insert is ligated into a vector sequence. If multiple vectors are present, this is less of a concern since they will all be degraded at the end of the process. In some embodiments, the vector possesses a tag that can be used to purify the vector away from the insert SMRTbell™ template product. In such embodiments, the vector sequences need not be degraded but can instead be reconstituted by removal of the stem-loops and addition of the portion “donated” to the insert SMRTbell™ template and reused. Alternatively, where the sizes of the vector SMRTbell™ templates and the insert SMRTbell™ templates are very different, size selection methods known in the art can be used to separate the vector SMRTbell™ templates from the insert SMRTbell™ templates to allow reuse of the vector. These separation methods, either tag- or size-mediated, may be preferred where it is difficult to find three different restriction enzymes that do not cleave the insert, e.g., where the insert is very large.

### Methods for Adding Priming Sites to Nucleic Acid Molecules

In certain aspects of the invention, nucleic acids for single-molecule sequencing are subjected to one or more manipulations so they can serve as templates for template-directed nascent strand synthesis, e.g., during polymerase-mediated sequencing by synthesis. For example, an oligonucleotide primer can be directly hybridized to a fragment to provide an initiation site for a polymerase enzyme. In addition, other “primer-free” methods for providing a site for polymerase binding and initiation of nascent strand synthesis include, but are not limited to, introducing nicks or gaps in the nucleic acid. Specific examples of certain of these methods are described in detail below.

Oligonucleotide primers can be random primers, or can comprise sequence complementary to a sequence in the template chosen by the practitioner. As noted above, they can comprise cognate nucleotides, non-cognate nucleotides, and nucleotides comprising one or more modifications, e.g., due to oxidative damage, methylation, alkylation, etc. Where the fragment/template is double-stranded, such primers can be invasive, e.g., comprising PNA or LNA. Such primers reduce or remove the need for potentially damaging heat-denaturation steps prior to primer annealing. In some embodiments, an oligonucleotide primer is designed to anneal to a portion of a template such that a polymerase initiating synthesis at the 3′ end of the primer will process a region of the template for which a nucleotide sequence is desired. In other embodiments, primers are generated by further fragmenting a portion of the sample nucleic acid. This is especially useful where the sequence of the sample nucleic acid is unknown. By fragmenting a portion of the sample, complementary primers are naturally generated. One preferred method for generating the primers from the nucleic acid sample is by treating an aliquot of the sample with DNaseI in the absence of Ca²⁺ and in the presence of Mg²⁺ to produce fragments less than 100 bp in length, which can be used to prime the bulk of the remaining nucleic acid sample. Additional methods of generating primers for priming a pool of nucleic acids, e.g., for sequencing-by-synthesis reaction, are provided is U.S. Ser. No. 12/553,478, filed Sep. 3, 2009 and incorporated herein by reference in its entirety for all purposes.

In yet further embodiments, where nucleic acid fragments are of unknown sequence, or the sequence is otherwise inappropriate for the design of oligonucleotide primers (e.g., such as in the case of highly repetitive nucleic acids), sequences (adapters) can be added to one or both ends of the fragments to provide priming sites. Addition of adapters can be accomplished by routine methods, such as ligation and, optionally, amplification techniques. For example, adapter sequences can be ligated to the ends of the fragments to provide known primer-binding sites. Such adapter sequences can be single- or double-stranded, or may comprise both single- and double-stranded portions. Like primers, they can comprise cognate, non-cognate, or modified nucleotides. Where an adapter sequence is fully double-stranded, helicase- or heat-assisted opening (denaturation) of the adapter can be used to provide a single-stranded binding site for a primer. In certain embodiments, adapters added to a double-stranded fragment are internally complementary such that they form hairpin structures upon conversion of the double-stranded fragment to a single-stranded fragment, e.g., where the 3′ end of the adapter sequence acts as a primer by folding back and annealing to provide an initiation site for the polymerase enzyme. In a related embodiment, an adapter can be added by treatment with a terminal transferase enzyme in the presence of a single type of nucleotide, e.g., dA to add a poly-A tail to the 3′-end of a fragment. Subsequently, a poly-T primer having a 3′-OH is hybridized to the poly-A tail, e.g., to provide a binding and initiation site for a polymerase enzyme.

The nucleic acid fragment can also be modified to provide a site for binding and initiation of nascent strand synthesis in the absence of exogenous primer sequences. Particular benefits of the “self-priming” templates include a much simplified and therefore more efficient sample preparation protocol. In such “primer-free” embodiments, there is no need to include a step for hybridizing primers to the templates prior to template-directed nascent strand synthesis, and the lack of a hybridization step also removes any inherent primer-hybridization bias, e.g., when multiple different primers with different characteristics (e.g., annealing temperatures, GC-contents, etc.) are used. Further, given that nucleic acid manipulations result in some loss of the nucleic acid sample, a sample prep method comprising fewer and less complex steps is not expected to suffer from as much loss of the sample, and therefore less sample can be used to carry out the experiment.

The types of modifications that provide a polymerase priming site vary depending on the requirements of the polymerase to be used, but generally include nicks and gaps, which provide a free 3′ end from which a polymerase can extend a nascent strand. Such a modification can be introduced into the nucleic acid fragment itself, or can be added to the fragment, e.g., by ligation of an adapter comprising the modification. For example, single-strand nicks can be introduced using various known molecular biology techniques, e.g., limited nuclease (e.g., DNasel) treatment and treatment with a nickase (e.g., Nt.BbvCI nicking endonuclease), where the sequence is known to contain a recognition site for a nickase. In other embodiments, a class of very short peptides containing the sequence SH (N-serine-histidine-COOH) exhibits DNA nicking activity that can be utilized in a very controllable fashion. (See, e.g., International Application No. PCT/US2001/043079, incorporated herein by reference in its entirety for all purposes.) In some embodiments, a nick is further resected to produce a gap of one or more nucleotides, as some polymerases prime more efficiently at a gap than at a nick. Resection of the nick to form a gap can be performed by various methods, e.g., limited exonuclease (e.g., T7 exonuclease) degradation, thermal degradation, and use of various, polymerases (e.g., T4 DNA polymerase or E. coli pol I). In yet further embodiments, a modification can be introduced by amplification using amplification primers comprising the modification, where the primers are complementary to the fragments and/or adapters ligated thereto. As such, the nucleic acid sample need only be fragmented, amplified to incorporate a site appropriate for modification, and treated with a modification agent to provide the site for initiation of polymerase-mediated nascent strand synthesis. In some embodiments, modification of the nucleic acid fragments comprises addition of a protein that facilitates polymerase initiation at an end. For example, Φ29 polymerase can initiate nascent strand synthesis from a dsDNA end in the presence of Φ29 terminal protein.

In some embodiments, splint oligos, described above, are used to create a nicked or gapped strand for primer-free sequencing of a single-stranded circular template. For example, a linear, single-stranded fragment can be treated to prevent ligation or strand extension from the 3′ end. A splint oligo used to circularize this fragment comprises terminal regions that are complementary to the ends of the fragment and a central region that is not complementary, such there is a gap between where the ends of the fragment will hybridize to the splint oligo. Extension of the splint oligo to generate a complementary strand and ligation to close the complementary strand are performed, and the 3′ end of the original single-stranded fragment is repaired to allow initiation of polymerase synthesis at the gap. Where incorporation of nucleotides is recorded during nascnet strand synthesis, the sequence read will correspond to the sequence of the original template, since the complement is acting as the template in this approach. In a similar embodiment, the splint brings the ends of the single-stranded fragment together, and a polymerase initiates from the nick present as a result of the non-ligatable 3′ end of the fragment. For primer-free initiation in a circular, double-stranded template, an adapter used to convert a linear, double-stranded fragment into the circular template can comprise a gap or nick, although a nick would need to be protected from the ligase activity required to attach the adapter to the ends of the fragment. Alternatively, a nick could be specifically introduced in the adapter using methods described elsewhere herein, and the nick could optionally be extended to form a gap.

As noted above, adapters having select characteristics can be attached to nucleic acid fragments in various ways. Where the ends of nucleic acid fragments are known, e.g., where fragmentation was performed using sequence-specific methods such as restriction endonuclease digestion, adapters can be designed to anneal to the ends of the fragments and to comprise a desired modification. Where the ends of nucleic acid fragments are unknown, e.g., where fragmentation was performed using sequence-nonspecific methods such as shearing, sonication, or heat fragmentation, adapters can be added to the ends of the fragments to provide known sequences to which primers can be designed. Alternatively, for fragments having overhangs, whether as a result of the fragmentation methodology or subsequent treatment, e.g., with a single-stranded exonuclease, a partially double-stranded adapter can be designed having a double-stranded portion comprising the modification desired to be added to the fragments, and a single-stranded portion that is complementary to the overhang. For nucleic acid preparations having random overhang sequences, the single-stranded portions could be randomly generated such that a plurality of adapters are created, each with a double-stranded portion comprising a modification and a random (or otherwise varied) single-stranded portion. The plurality of adapters could all comprise the same modifications, or different modifications could be present, e.g., depending on the sequence of the “random” portion, e.g., where the random portion of each adapter is known to the practitioner. The same primer can be added to both ends of a fragment, or different primers can be added to each end, e.g., one with a modification and one without the modification, or both having the same or different modification. Any of these methods would provide a primer or primer binding site to facilitate polymerase initiation, e.g., for amplification or sequencing of the nucleic acid fragment.

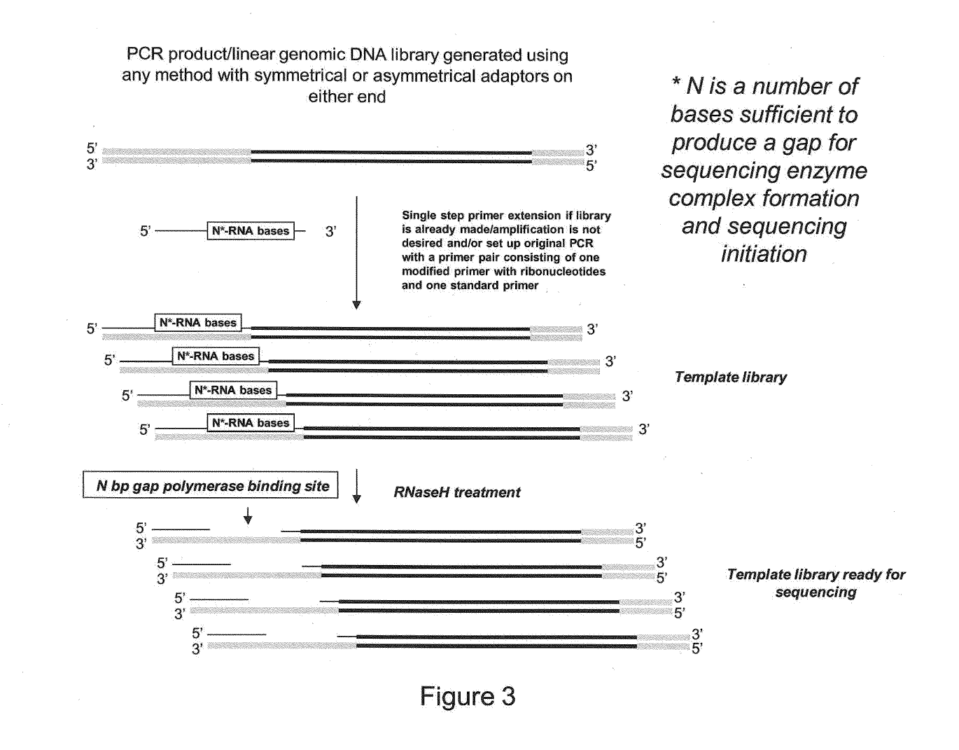

In some embodiments, single-stranded adapters are used to modify a linear, double-stranded fragment to provide for primer-free initiation of nascent strand synthesis. For example, the modification can be a region of ribonucleotides (e.g., two or more) such that the resulting amplicons comprise the ribonucleotides within an otherwise deoxyribonucleotide composition, as shown in FIG. 3. The fragments are subsequently treated with a ribonuclease, e.g. RNaseH, to degrade the ribonucleotides, thereby creating a single-stranded region within the otherwise double-stranded template molecule. The size of the single-stranded region is dependent on the number of ribonucleotides present prior to digestion, and this number can be optimized for a particular polymerase enzyme based upon the known characteristics of that enzyme. For ease of discussion, the strand from which the ribonucleotides are removed is termed the “gapped strand” and the opposite strand is termed the “ungapped strand.” A polymerase enzyme binds to the single-stranded region and extends the 3′-OH to synthesize a strand complementary to the ungapped strand. Preferably, the polymerase comprises strand-displacement activity to remove the gapped strand as it synthesizes a complement to the ungapped strand. One benefit of the single-step primer extension is that it allows direct sequencing of the original nucleic acid fragment because it is contained, without modification, within the strand that serves as the template; therefore, any modifications (e.g., methylated or hydroxymethylated bases) originally in the sample nucleic acid fragment are processed during the sequencing reaction. Methods for detection modified bases during sequencing are described in International Application No. PCT/US2011/060338, filed Nov. 11, 2011, the disclosure of which is incorporated herein by reference in its entirety for all purposes. Other types of modifications can also provide a site for initiation of polymerization, e.g. nicking sites, and the invention is therefore not limited by the type of modification used to facilitate polymerase binding and strand extension. For example, glycosylases specific for various nucleotide modifications exist, and treatment of such a modification with the appropriate glycosylase followed by treatment with an AP endonuclease produces a nick in place of the modification. Although the scheme in FIG. 3 includes a single-step primer extension, the modified primers can also be used in amplification reactions, e.g., in combination with standard primers.

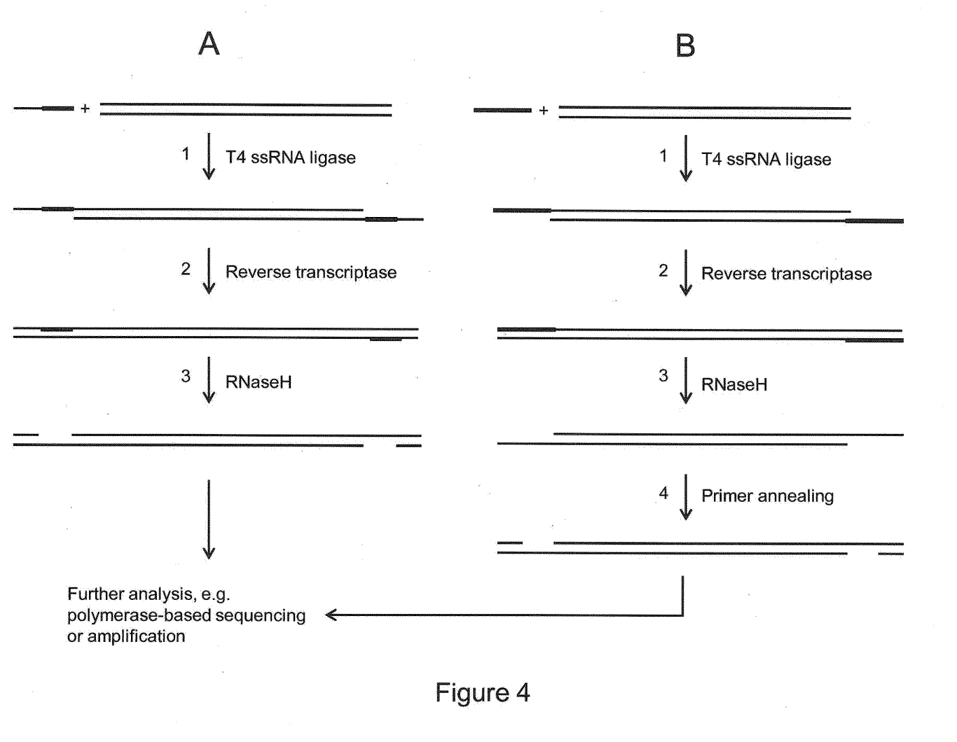

In related embodiments, a single-stranded adapter having a 3′-hydroxyl group on a ribonucleoside at the 3′ terminus can be attached to an end of a double-stranded fragment having 5′-phosphoryl groups by T4 ssRNA ligase. In certain embodiments, adapters comprising one or more 3′-terminal ribonucleotides appropriate for T4 ssRNA ligation and a region of 5′ terminal deoxyribonucleotides sufficient for polymerase binding are ligated to the ends of a double-stranded fragment with T4 ssRNA ligase, as shown in FIG. 4A, step 1. Extension of the unligated ends of the fragment using one or more polymerases, e.g., an RNA-dependent DNA polymerase (e.g., reverse transcriptase) and a DNA-dependent DNA polymerase, results in a blunt-ended double-stranded molecule having internal regions of one or more ribonucleotides (FIG. 4A, step 2). As in the embodiment above, treatment with RNaseH results in removal of the ribonucleotides, leaving a gap in their place (FIG. 4A, step 3). The gap can be used as an initiation site of the polymerase, which will extend from the 3′-end of the DNA region of the adapter. A similar primer-dependent strategy comprises ligation of an RNA adapter that does not comprise deoxyribonucleotides to the ends of a double-stranded fragment (FIG. 4B, step 1), extension of the unligated end of the fragment using an RNA-dependent DNA polymerase (FIG. 4B, step 2), digestion of the RNA adapter (FIG. 48, step 3), and addition of primers complementary to the extended ends of the fragment to provide a polymerase initiation site (FIG. 4B, step 4). Although RNaseH is used to remove the ribonucleotides in the methods above, certain glycosylases can also be used. For example, uracil-DNA glycosylase removes uracil bases to create abasic sites, and AP endonuclease can be subsequently added to remove the remaining sugar and phosphate to produce a single-base gap in the strand for every uracil that was previously present. That is, three adjacent uridine monophosphates are converted to a three base gap.

In further embodiments, a terminal transferase enzyme is used to modify a linear, double-stranded nucleic acid to provide a site for initiation of polymerase-mediated strand synthesis. As noted elsewhere herein, the linear, double-stranded nucleic can be the result of fragmentation of larger (e.g., genomic) nucleic acids produced by shearing or restriction digest (or other nuclease reaction). In preferred methods, a terminal deoxynucleotidyl transferase (e.g., TdT) is used to add a tail to the 3′ ends of the double-stranded molecule. The terminal transferase is provided a first type of nucleotide (e.g., dA), and after a length of poly-A has been added a large spike of the complementary nucleotide (e.g., dT) is added to the reaction. The addition of the complementary nucleotide is carried out for a shorter time than that of the first nucleotide to ensure that the length of the sequence of complementary nucleotides is shorter than the length of the sequence of first nucleotides. The resulting tail is composed of two regions that are complementary to each other (e.g., poly-A and poly-T), and therefore will fold back upon itself to form a hairpin having a 3′-OH positioned to serve as a primer for a polymerase enzyme to process a strand of the double-stranded nucleic acid. Since the length of the sequence of complementary nucleotides is shorter than the length of the sequence of first nucleotides, the 3′-OH will be next to a gap, which is a preferred initiation site for certain polymerase enzymes.

In alternative embodiments, the above-described method begins with a linear, single-stranded nucleic acid rather than a double-stranded nucleic acid. The addition of the self-complementary tail occurs at only the 3′ end, and annealing of the tail to itself (e.g., poly-A/poly-T) provides a binding and initiation site for a polymerase enzyme, which extends the tail by synthesizing a complement to the original single-stranded nucleic acid. Optionally, after synthesis of the nascent strand, a second tail can be added to its 3′ terminus to be used to prime a second nascent strand that is complementary, in the following order, to the first nascent strand, the first tail, and finally the original single-stranded nucleic acid. As such, identifying sequential bases incorporated into the first nascent strand provides a sequencing read complementary to the single-stranded nucleic acid; and identifying sequential bases incorporated into the second nascent strand provides a sequencing read complementary to the first nascent strand, the first tail, and finally the original single-stranded nucleic acid. Identification of bases incorporated into a nascent strand during polymerase-mediated strand synthesis is further described below and elsewhere herein.

In preferred embodiments, such templates are used for polymerase-mediated sequencing by synthesis, in which incorporation of nucleotides into a nascent strand is monitored to determine a sequence of base incorporations that is indicative of the nascent strand and, by complementarity, the template strand. Some such methods are provided in the art, e.g., in Eid, et al. (2009) Science 323:133-138; Levene et al. (2003) Science 299: 682-686; Korlach, et al. (2008) Nucleotides, Nucleosides, and Nucleic Acids 27:1072-1083; Travers, et al. (2010) Nucl. Acids Res. 38(15):e159; Korlach, et al. (2010) Methods in Enzymology 472:431-455; and U.S. Pat. Nos. 7,315,019 and 7,056,661, the disclosures of all of which are incorporated herein by reference in their entireties for all purposes. Although the templates provided herein are particularly suitable for single-molecule, real-time sequencing technologies, they are also applicable to other technologies that require polymerase binding and nascent strand synthesis, including but not limited to SOLiD sequencing from Life Technologies Corp. (Carlsbad, Calif.), pyrosequencing from 454 Life Sciences (Branford, Conn.), tSMS from Helicos BioSciences (Cambridge, Mass.), and Solexa sequencing from Illumina, Inc. (San Diego, Calif.).

Methods and Compositions for Generating Sequencing Templates Comprising cDNA Synthesized from a Full-Length mRNA Molecule

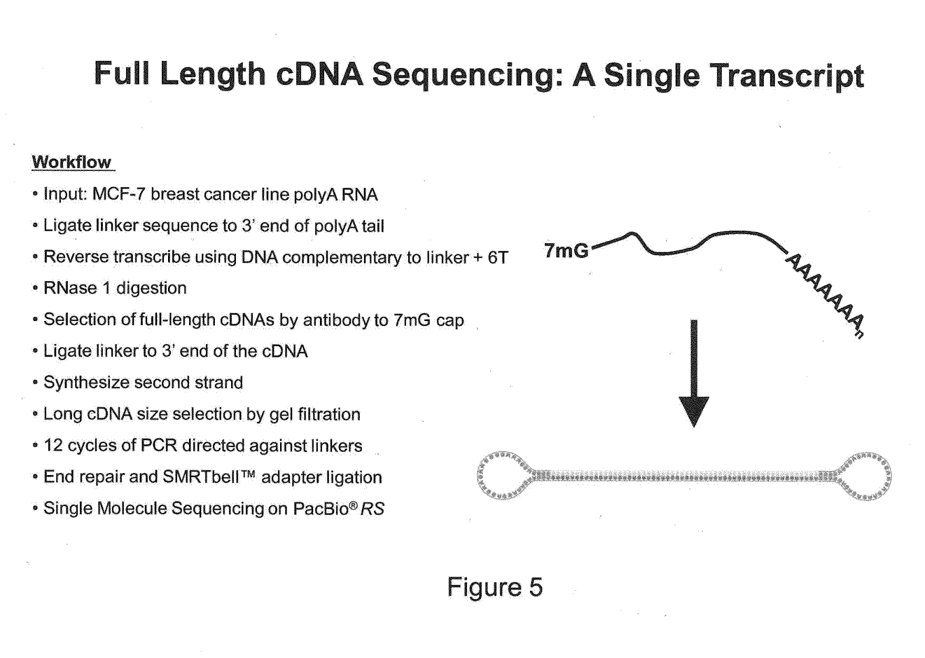

In certain aspects, the invention provides methods and compositions for generating a template for template-directed DNA sequencing from a full-length mRNA molecule (including the poly-A tail.) An exemplary embodiment is shown in FIG. 5 and described below.

In cells, the 3′ end of eukaryotic messenger RNAs (mRNAs) is polyadenylated in a post-transcriptional fashion by a series of enzymatic events beginning with mRNA cleavage, then processive adenylation by a specific polymerase. In metazoans, these tails are thought to be over 100 nucleotides in length in most cases. The poly-A tail plays a critical role in the stability of the transcript and it also regulates the occupancy of the mRNAs in the translating ribosomes. In general, polyadenylation means stability for a transcript and deadenylation leads to rapid degradation.

Polyadenylation has been previously studied on a gene-by-gene basis due to the difficulty in measuring the length of the poly-A tail by conventional means other than direct hybridization methods. Reverse transcription/PCR cloning of poly-A tails into bacteria for subsequent Sanger sequencing results in unreliable sequencing due to the homopolymeric nature of the sequence. Other kits use tailing of the poly-A tail with another homopolymer stretch and subsequent RT-PCR with a gene-specific forward primer and a reverse primer against the new homopolymer stretch. Again, this makes measurement possible but it is only amenable to a gene-by-gene analysis and it depends upon knowledge of sequence upstream of the poly-A tail for design of the forward primer.

Recent findings in the field of alternative 3′ end formation indicate that the length and sequence of the 3′ untranslated region (3′-UTR) may play important roles in cancer, regulation by microRNAs, and regulation of poly-A tail length. As such, there is a need for a method capable of simultaneously determining the length of the poly-A tail and the identity of the 3′-UTR to which it is attached. Certain aspects of the invention provide such methods.

In preferred embodiments, mRNA molecules are removed or purified from a sample source (e.g., cell culture, tissue sample, etc.), and a 5′-phosphorylated linker having a defined sequence is ligated to each of the 3′ termini of the poly-A tails. This defined sequence provides a primer site for reverse transcription and PCR. Optionally, it can include other sequence elements, e.g., restriction sites, modified bases, registration sequences or “tags,” structural moieties, or other modifications. Ligation is typically performed with the linker annealed to a biotinylated DNA oligo that also comprises a 3′ poly-T overhang to base pair with the 3′-end of the mRNA poly-A tail. The poly-T overhang makes ligation more efficient and discriminating, e.g., between poly-A RNAs and other RNAs. In certain embodiments, T4 RNA Ligase 2 (New England Biolabs, Ipswich, Mass.) is a preferred ligase for this reaction. It is known that some mRNAs have poly-U nucletides at their 3′ ends, and it is further contemplated that different overhang sequences on the oligo may be utilized to capture such mRNA templates.

The mRNA is reverse transcribed using the biotinylated oligo to prime the synthesis of a DNA complement to the mRNA transcript, where the nascent strand is synthesized by extension of the poly-T overhang. In preferred embodiments, reverse transcription is accomplished using a reverse transcriptase that is free of RNase activity (e.g., M-MLV reverse transcriptase), and is optionally thermostable. The resulting cDNA is complementary to the full-length transcript. The mRNA is subsequently digested, e.g., using RNaseH or another appropriate RNA-specific nuclease, and, optionally, full-length cDNA (i.e., comprising sequence complementary to all or substantially all of the mRNA transcript) is selected, e.g., using an antibody or other protein specific to the 7 mG cap (e.g., eukaryotic translation initiation factor 4E, or eIF4E). A linker is ligated to the 3′ end of the newly synthesized cDNA strand, and a complementary DNA strand is synthesized to generate a double-stranded cDNA molecule, e.g., by annealing a primer to the linker and performing a primer extension reaction. Similar to the linker ligated to the 3′ end of the mRNA transcript, this linker can include other sequence elements, e.g., restriction sites, modified bases, registration sequences or “tags” (e.g., to identify the transcript being sequenced), structural moieties, or other modifications. The full-length double-stranded cDNA molecule can be optionally selected using an antibody or other protein specific to the 7 mG cap to isolate full-length cDNA products of the reverse transcription reaction. In other embodiments, a size selection by gel filtration can be performed to select for long or full-length double-stranded cDNA products. Alternatively, the biotin on the oligo complementary to the 3′ linker can be used to isolate the double-stranded product. In certain embodiments a gel filtration-based size selection is performed to isolate full-length, fully double-stranded DNA products, e.g., that were selected using the biotin tag.

Where it is desirable to sequence the full-length cDNA, the selected molecules can be directly sequenced, or can be optionally amplified prior to sequencing. Such an amplification is typically directed against the linkers at each end. The full-length double-stranded cDNA molecules, optionally amplified, are used to synthesize SMRTbell™ templates, which are closed linear constructs comprising a central double-stranded portion flanked by stem-loop adapters such that upon separation or “melting” of the double-stranded portion a circular, single-stranded molecule is produced, as described elsewhere herein. In the absence of amplification, portions of the linkers at the ends of the molecules can be removed, e.g., by restriction digestion. In particular embodiments, the 3′ linker-oligo construct comprises a restriction site that allows cleavage of the biotin tag from the molecule. Further description of such templates is provided in U.S. patent application Ser. No. 12/413,258, filed Mar. 27, 2009, and incorporated herein by reference in its entirety for all purposes. This template is suitable for template-directed sequencing, in particular single-molecule real-time sequencing as provided by Pacific Biosciences of California, Inc. and as further described, e.g., in Eid, et al. (2009) Science 323:133-138; Levene, et al. (2003) Science 299: 682-686; Korlach, et al. (2008) Nucleotides, Nucleosides, and Nucleic Acids 27:1072-1083; Travers, et al. (2010) Nucl. Acids Res. 38(15):e159; Korlach, et al. (2010) Methods in Enzymology 472:431-455; and U.S. Pat. Nos. 7,315,019 and 7,056,661, the disclosures of all of which are incorporated herein by reference in their entireties for all purposes. These methods typically use a single polymerase enzyme to sequence an entire sequencing template in a processive manner, and detect incorporation in during the reaction, e.g., using optically detectable nucleotide substrates. In addition, such single-molecule real-time sequencing methods are capable of producing long sequencing reads, e.g., at least about 100, 150, 200, 500, 1000, 5000, 10,000 bases or longer. As such, full-length mRNA sequence can be generated in a single sequencing read, e.g., by the action of a single polymerase enzyme on a single cDNA sequencing template (e.g., a SMRTbell™ template comprising a full-length cDNA sequence).

Where it is desirable to sequence only a portion of an mRNA, e.g., the poly-A tail and, optionally, the 3′ UTR, the selected molecules can be subjected to a restriction digestion with a frequent cutter that does not have a restriction site within the poly-A tail and, optionally, the 3′-UTR (e.g., MboI). Restriction enzymes and their specificities are well known to the ordinary practitioner, and many different types and combinations thereof can be used to fragment the full-length cDNA molecule. Where a biotin tag is present at the portion of the cDNA corresponding to the poly-A tail of the mRNA, this tag can be used to isolate fragments comprising the poly-A tail and, where present, the adjacent 3′-UTR. Preferably, the biotin (or other) tag is removed subsequent to the isolation, e.g., by restriction digestion as described above. The resulting fragments can be integrated into sequencing templates (e.g., SMRTbell™ templates), as described above.

In certain preferred embodiments, poly-A mRNA is provided by methods routine in the art A linker sequence is ligated to the 3′ end of the poly-A tail, and a biotinylated DNA oligonucleotide having a six-base poly-T 3′ overhang is hybridized to the linker and the terminal six A bases in the poly-A tail. Reverse transcription is performed, followed by second strand synthesis to generate a double-stranded cDNA molecule. A subsequent restriction digest (e.g., with MboI or MspI) fragments the double-stranded cDNA, and the 3′ ends are recovered by binding to streptavidin beads. The recovered cDNA fragments are ligated to stem-loop adapters to form SMRTbell™ templates. This method requires no amplification step, and the average size of the cDNA fragments captured is ˜190 bp.

### EXAMPLES

### OTUB1 mRNA

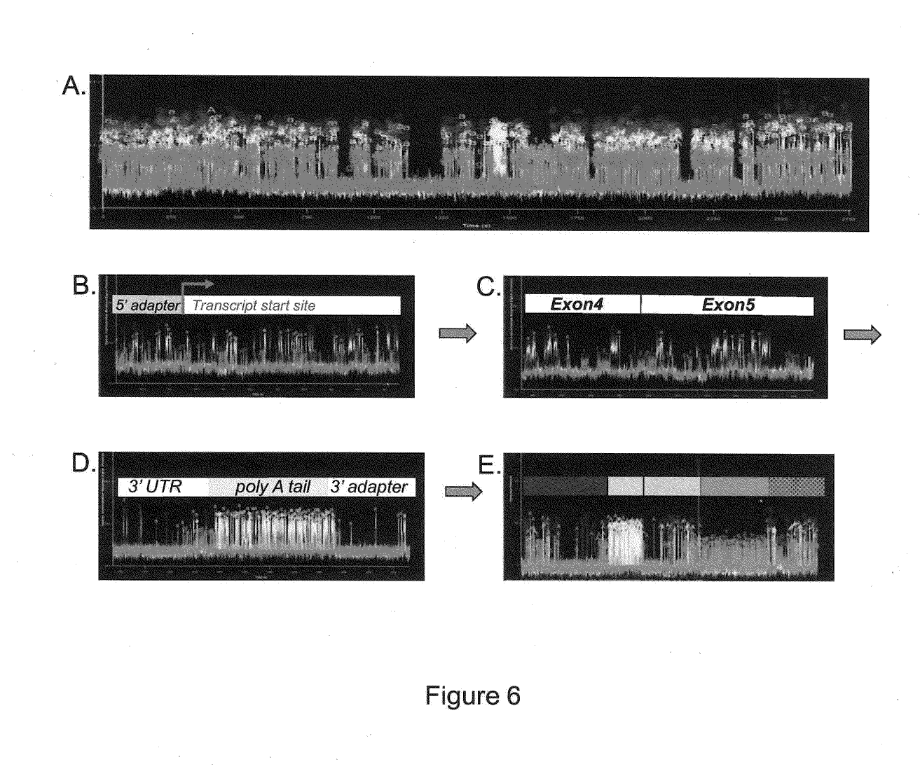

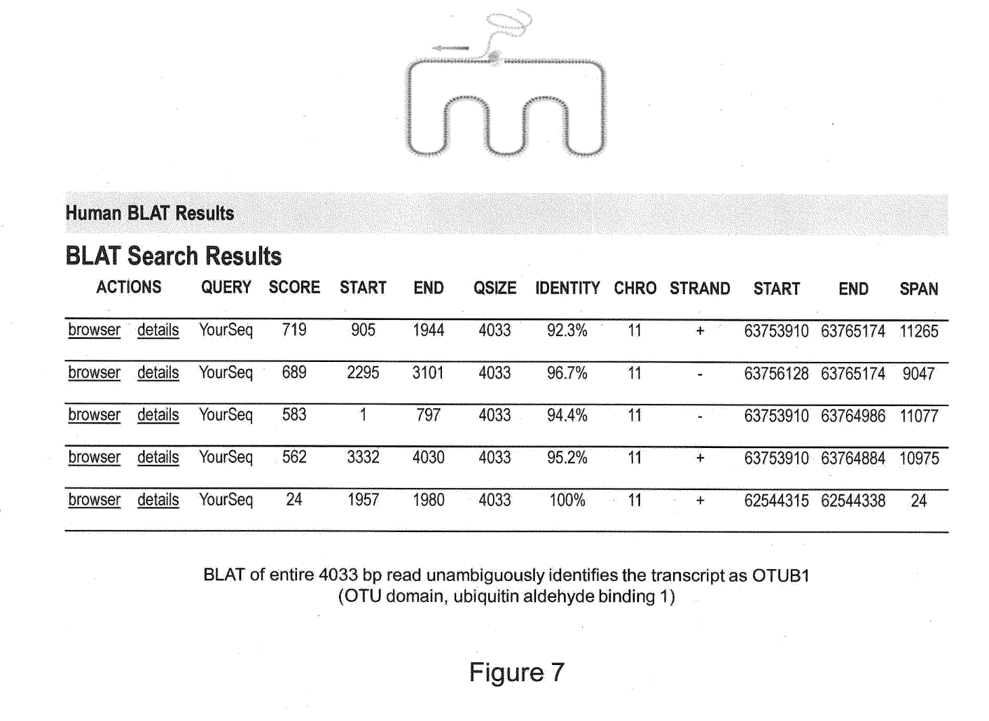

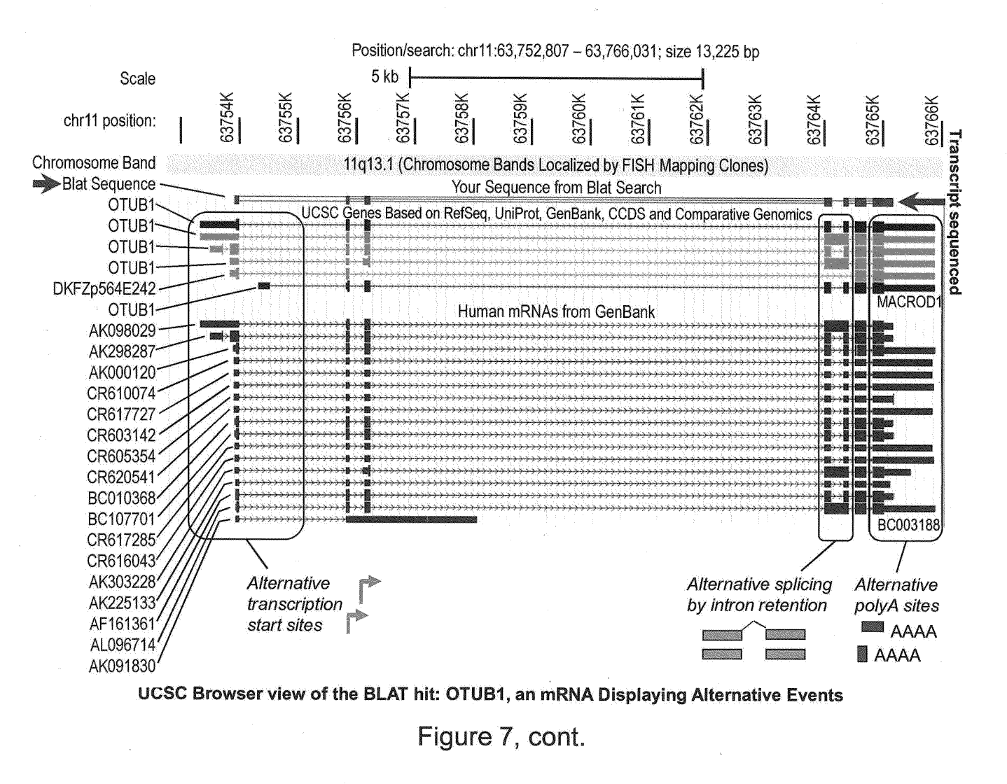

Data from single-molecule, real-time, polymerase-mediated sequencing reactions using SMRTbell™ templates is shown in FIG. 6. Panel A illustrates a sequencing “trace” during a thirty minute real-time single-molecule polymerase-mediated sequencing-by-synthesis reaction. Peaks in the trace correspond to incorporation events during the reaction. Panel B shows a portion of the trace comprising the 5′ adapter adjacent to the start site of the OTUB1 transcript, demonstrating that OTUB1 transcript began at a downstream start site in the gene. Panel C illustrates that the trace comprises the sequences for exon 4 and exon 5 adjacent to one another, demonstrating that the intervening intron had been spliced out during mRNA post-transcriptional processing. Panel D illustrates the portion of the trace comprising the 3′ UTR, the poly-A tail, and the 3′ adapter, and revealed 111 distinct A pulses, indicating that the poly-A tail of the mRNA comprised 111 adenosine nucleotides. Panel E illustrates that the sequencing trace continues through the 3′ adapter and into the complementary strand, passing through sequence complementary to the poly-A region (the poly-T stretch) and 3′ UTR regions shown in panel D. As such, this sequencing data identified regions that were spliced out of the mRNA transcript, and was able to measure the 111 adenine bases in the poly-A tail. The sequence read data from the reactions shown in FIG. 6 was used to unambiguously identify the mRNA transcript as OTUB1 (OUT domain, ubiquitin aldehyde binding 1) based upon a BLAT search of publicly available sequence information (FIG. 7).

DSCAM mRNA

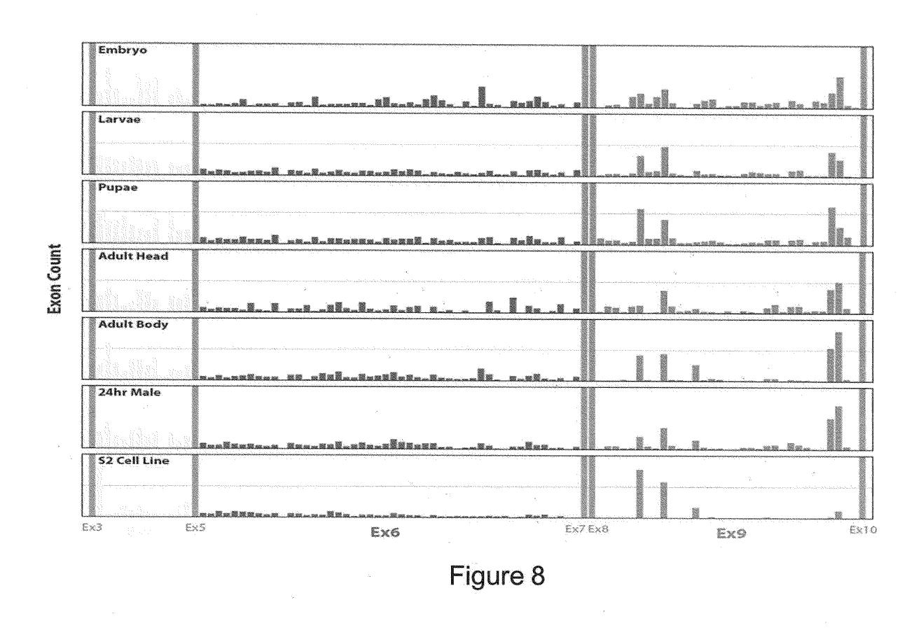

The Drosophila DSCAM gene has 24 exons and the DSCAM mRNA is 7.8 kb in length and has 38,016 possible isoforms. However, previous studies have not revealed how many of these possible isoforms are actually expressed, nor whether or how expression might change depending on various factors including, but not limited to, tissue type, developmental stage, presence of drugs, etc. Single-molecule, real-time, polymerase-mediated sequencing reactions were used to generate the data shown in FIG. 8, which provides DSCAM exon representation in seven different tissues in Drosophila: embryonic, larval, pupal, adult head, adult body, two-day-old male, and the Schneider 2 (S2) cell line. These data showed clear differences in mRNA isoform expression that were tissue-dependent. Interestingly, the S2 cell line had a significantly different expression pattern that did the embryonic tissue even though the S2 cell line is derived from embryonic cells. Importantly, this read method can collectively view which combination of splice site choices were made on the same mRNA transcript.

### Poly-A Tail Homopolymer Sequencing

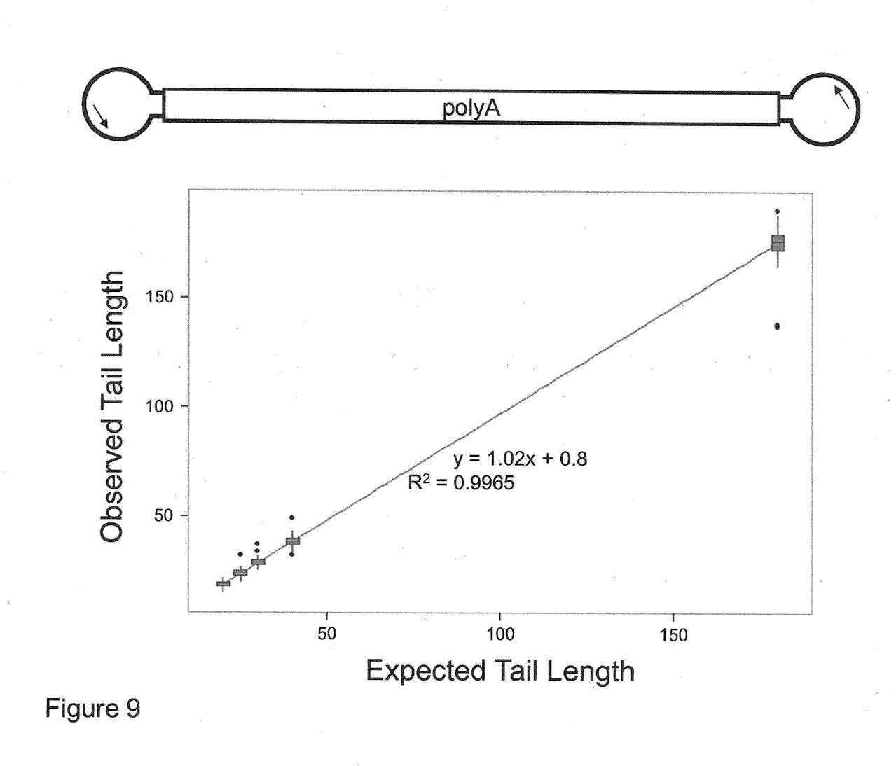

Eukaryotic poly-A tails can range in length from tens to several hundreds of adenosine nucleotides. SMRT® sequencing analysis was carried out using sequencing templates having poly-A sequences of different defined lengths: 20, 25, 30, 40, and 180 adenine bases. FIG. 9 illustrates the sequencing results, demonstrating that this sequencing method was able to accurately estimate the number of adenosine nucleotides in each of the five different templates.

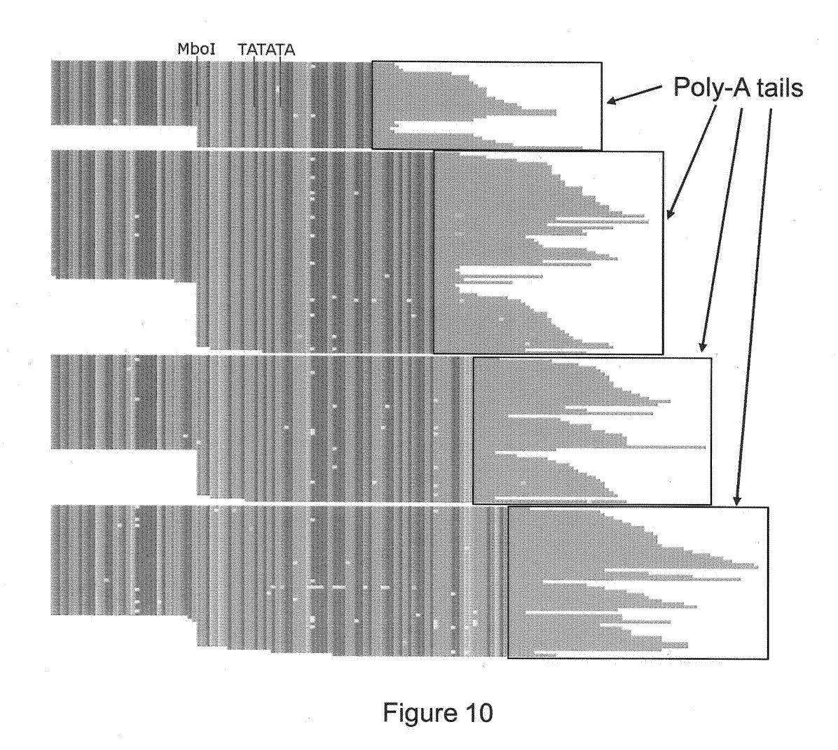

Yeast RPS12 (systematic name YOR369C) encodes a protein component of the small (40S) ribosomal subunit. Sequencing of the mRNA products of this gene revealed that they have more than one polyadenylation site and multiple different poly-A tail lengths at each of those sites. Data from this study is illustrated in FIG. 10.

β-Actin mRNA

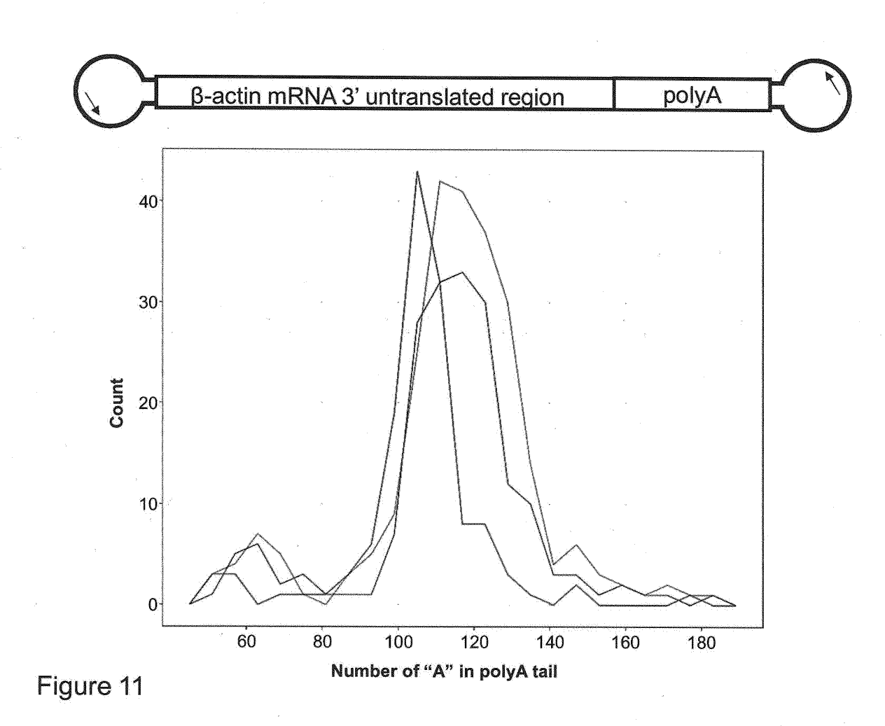

In a separate study, the poly-A tail of β-actin mRNA was measured using SMRT® sequencing as described herein. FIG. 11 illustrates that the measured length of the poly-A tail corresponded to the size estimate derived from a gel-based size determination assay (˜120 adenosine nucleotides).

Poly-A Tails and UTRs from S. Cerevisiae mRNA

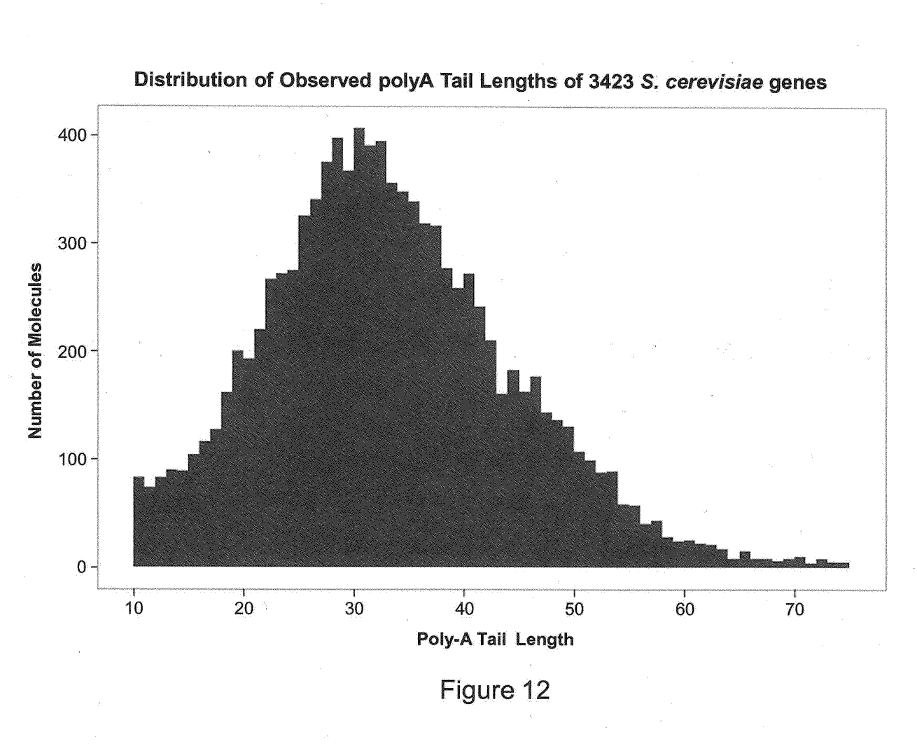

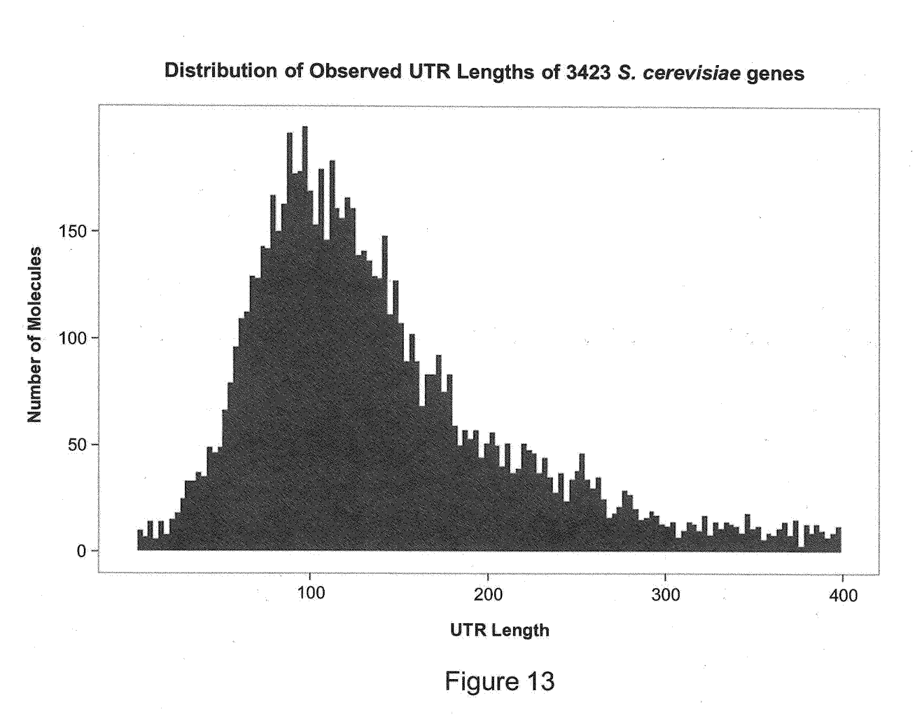

In a further study, a distribution of poly-A tails from 3423 genes in S. cerevisiae was measured using SMRT® sequencing. Briefly, mRNA was isolated from an S. cerevisiae culture using standard molecular biology methods. A linker sequence was ligated to the 3′ end of the poly-A tail and a reverse transcriptase was used to synthesize a complementary DNA strand from a biotinylated DNA primer having a 5′ portion complementary to the linker and a 3′ portion complementary and specific to the six terminal adenine nucleotides of the poly-A tail. RNase treatment degraded the original mRNA, and a DNA strand complementary to the strand synthesized by the reverse transcriptase was generated using a DNA-dependent DNA polymerase. The resulting double-stranded molecules were fragmented with a restriction endonuclease (Mbol or Mspl), and the fragments covalently linked to the biotinylated primer at the 3′ end were captured by binding to streptavidin. These captured fragments were incorporated into SMRTbell™ templates and sequenced. A graphical representation of the resulting poly-A length distribution from the 3423 S. cerevisiae genes is provided in FIG. 12. Connected to the poly-A tails were the untranslated regions (UTRs) of the same 3423 S. cerevisiae genes, identified via the same sequencing reads. The distribution of the UTR lengths is provided in FIG. 13. This distribution is consistent with that previously predicted from EST and ORF data, as described in Graber, et al. (1999) Nucl. Acids Res. 27(3):888-94, incorporated herein by reference in its entirety for all purposes.

### Newly Discovered Non-Coding Transcript Having an 84 bp Poly-A Tail

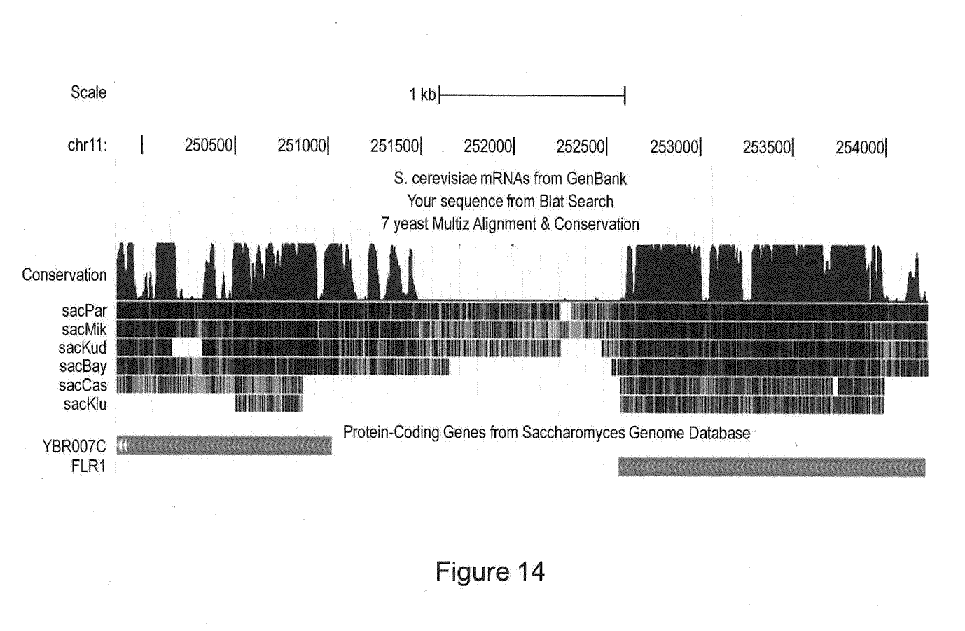

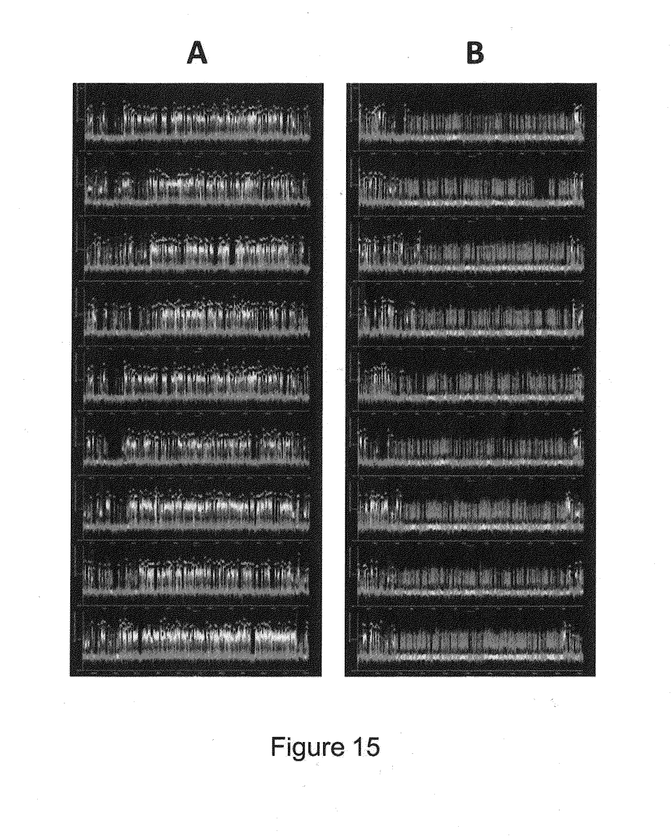

A previously unidentified non-coding RNA transcript was also discovered in another cDNA sequencing study. FIG. 14 shows that a polyadenylated transcript appears between two known yeast transcripts. The arrows show the direction of transcription of this RNA, which has a poly-A tail of 84 nucleotides. Interestingly, this RNA is transcribed antisense to the known mRNAs that flank it, i.e., it is transcribed from the opposite strand as two known neighboring genes. The sequencing traces are provided in FIG. 15 and represent nine passes around a closed nucleic acid construct comprising a cDNA for the transcript. FIG. 15A provides the portions of the trace corresponding to the strand having the poly-A tail, and 15B provides the portions of the trace corresponding to the complementary strand. The presence of a poly-A tail in this transcript is indicative that this RNA is most likely transcribed by Pol II, and it could represent an antisense transcript to a neighboring gene, a phenomenon known to serve a regulatory role in some cases.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of producing a double-stranded nucleic acid having a single-stranded region, the method comprising:
a) providing a double-stranded DNA molecule;
b) fragmenting the double-stranded DNA molecule to produce double-stranded DNA fragments;
c) attaching polynucleotides to the ends of the double-stranded DNA fragments, wherein at least one of the polynucleotides on each of the fragments comprises a region of ribonucleotides; and
d) eliminating the region of ribonucleotides to produce a double-stranded nucleic acid having a single-stranded region.

2. The method of claim 1, wherein the eliminating is performed using a ribonuclease.

3. (canceled)

4. The method of claim 1, wherein the attaching comprises performing a single-step primer extension from a primer comprising the region of ribonucleotides.

5. The method of claim 1, wherein the attaching comprises ligating an adapter comprising the region of ribonucleotides.

6. The method of claim 5, wherein the adapter further comprises a region of deoxyribonucleotides that is terminally located after the attaching.

7. The method of claim 5, wherein the adapter is a single-stranded adapter that is ligated to 5′ ends of both strands of the double-stranded DNA fragments, and the method further comprises performing a strand extension reaction to extend 3′ ends of both strands of the double-stranded DNA fragments, thereby converting the single-stranded adapter to a double-stranded adapter.

8-12. (canceled)

13. A method of producing a nucleic acid template, the method comprising:
a) providing a nucleic acid molecule comprising a region of interest;
b) digesting the nucleic acid molecule to provide a mixture comprising a fragment of the nucleic acid molecule comprising the region of interest and one or more additional fragments of the nucleic acid molecule that do not comprise the region of interest;
c) ligating hairpin adapters to the ends of the fragment and the ends of the additional fragments;
d) performing a second digestion of the additional fragments wherein the fragment comprising the region of interest is not cleaved, thereby converting the additional fragments into substrates for exonuclease activity; and
e) subjecting the mixture to an exonuclease digestion, thereby digesting the additional fragments while not digesting the fragment comprising the region of interest, thereby synthesizing a nucleic acid template comprising the region of interest.

14-22. (canceled)

23. A method of generating a cDNA sequencing template from a full-length mRNA transcript comprising ligating a first linker onto the 3′ end of a poly-A tail; synthesizing a DNA complement to the mRNA transcript; degrading the mRNA transcript; generating a complement to the DNA complement to the mRNA transcript, thereby producing a double-stranded cDNA molecule appropriate to serve as a template nucleic acid in a polymerase-mediated sequencing-by-synthesis reaction.

24. The method of claim 23, wherein the full-length mRNA transcript is at least 100 base pairs in length.

25. (canceled)

26. The method of claim 23, wherein the first linker comprises a sequence complementary to a first primer used in the synthesizing of the DNA complement to the mRNA transcript.

27. The method of claim 26, wherein the first primer comprises a poly-T region at its 3′ end.

28. The method of claim 26, wherein the first primer is biotinylated.

29. (canceled)

30. The method of claim 23, further comprising selecting for cDNA comprising sequence complementary to the full-length mRNA transcript based upon the presence of sequence complementary to a 7 mG cap of the full-length mRNA transcript.

31. The method of claim 23, wherein the generating comprises ligating a second linker to a 3′ end of the DNA complement to the mRNA transcript, wherein the second linker serves as a binding site for a primer, and wherein the primer serves as an initiation site for a primer extension reaction.

32. The method of claim 23, further comprising ligating the double-stranded cDNA molecule to two stem-loop adapters, thereby constructing a nucleic acid molecule having no free 3′ or 5′ ends.

33. A method of sequencing an mRNA transcript, the method comprising ligating a first linker onto the 3′ end of a poly-A tail region of a full-length mRNA transcript; synthesizing a DNA complement to the full-length mRNA transcript; degrading the full-length mRNA transcript; generating a complement to the DNA complement to the full-length mRNA transcript, thereby producing a double-stranded cDNA molecule; ligating the double-stranded cDNA molecule to two stem-loop adapters to generate closed nucleic acid constructs having no free 3′ or 5′ ends; and sequencing the closed nucleic acid constructs.

34. The method of claim 33, wherein the full-length mRNA transcript comprises both a poly-A tail region and a 7 mG cap.

35. The method of claim 33, wherein the full-length mRNA transcript is at least 100 base pairs in length.

36. (canceled)

37. The method of claim 33, wherein the first linker comprises a sequence complementary to a first primer used in the synthesizing of the DNA complement to the full-length mRNA transcript.

38. The method of claim 33, wherein the first primer comprises a poly-T region at its 3′ end.

39. The method of claim 33, wherein the first primer is biotinylated.

40. The method of claim 33, wherein the generating comprises ligating a second linker to a 3′ end of the DNA complement to the mRNA transcript, wherein the second linker serves as a binding site for a primer, and wherein the primer serves as an initiation site for a primer extension reaction.

41. The method of claim 33, further comprising fragmenting the double-stranded cDNA molecule to produce cDNA fragments, and selecting the cDNA fragments comprising a portion comprising the poly-A tail region of the mRNA transcript.

42-43. (canceled)

44. The method of claim 33, wherein said sequencing of the closed nucleic acid constructs provides sequence reads that encompass an entire sequence for the full-length mRNA transcript.

45. The method of claim 33, wherein said sequencing of the closed nucleic acid constructs is performed iteratively such that the closed nucleic acid constructs are sequenced processively at least twice by a single polymerase enzyme.

46. The method of claim 33, wherein said sequencing of the closed nucleic acid constructs is performed using a single-molecule, real-time sequencing method.
